(19) United States
(12) Patent Application Publication
(10) Pub. No.: US 2006/0134204 A1
(43) Pub. Date: Jun. 22, 2006

(54) Complexes made using low solubility drugs

(76) Inventors: Patrick S.L. Wong, Burlingame, CA (US); Dong Yan, Cupertino, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/301,832

(22) Filed: Dec. 12, 2005

Related U.S. Application Data
(60) Provisional application No. 60/638,273, filed on Dec. 21, 2004.

(51) Int. Cl. A61K 9/20

(52) U.S. Cl. 424/464

(57) ABSTRACT

Disclosed herein are substances that include a complex that includes a drug moiety ionically bound to a counter-ion; wherein a solubility of the complex is greater than a solubility of the drug moiety. Also disclosed are compositions and dosage forms made from such substances and complexes; methods of making compositions, substances, and complexes; and methods of administering the same to patients.

### CROSS-REFERENCE TO RELATED APPLICATIONS

This application claims priority to U.S. Provisional Application Ser. No. 60/638,273, filed on Dec. 21, 2004, and entitled “Complexes Made Using Low Solubility Drugs” incorporated by reference.

### BACKGROUND OF THE INVENTION

1. Field of the Invention

Disclosed herein are substances that include a complex that includes a drug moiety ionically bound to a counter-ion; wherein a solubility of the complex is greater than a solubility of the drug moiety. Also disclosed are compositions and dosage forms made from such substances and complexes; methods of making compositions, substances, and complexes; and methods of administering the same to patients.

2. Background Art

Lead compounds that are currently being developed using combinatorial chemistry and other high throughput techniques often demonstrate very poor solubility. This may be in part because pharmaceutical companies may choose to screen first for activity against a target, and only then for pharmacokinetic properties. This can lead to discovery of very active compounds that are not particularly good orally dosed drugs.

If new drug leads have poor solubility, this may lead to poor oral absorption from the gastrointestinal tract. Poor oral absorption leads to poor bioavailability, and consequently poor drug performance.

These problems have been recognized in the industry. See M. Kataoka et al., “In Vitro System to Evaluate Oral Absorption of Poorly Water-Soluble Drugs: Simultaneous Analysis on Dissolution and Permeation of Drugs,” Pharm. Res. 20(10):1674-1680 (2003). This document, and all others cited to herein, are incorporated by reference for all purposes as if reproduced fully herein.

Development of new technologies to improve solubility has generated scientific interest, resulting in a large array of new systems that can be applied to compounds with intrinsically low solubility. K. R. Horspool et al., “Advancing new drug delivery concepts to gain the lead.” Drug Delivery Technology 3:3446 (2003) (“Horspool”). Horspool goes on to say:

“Many of the systems have been designed to overcome solubility issues associated with high lipophilicity. However, problems remain with solubility associated with highly crystalline materials that exhibit strong intermolecular interactions and a high propensity to crystallize. This issue is exacerbated because discovery screening typically involves testing of amorphous forms of compounds in dimethyl sulphoxide (DMSO). Testing of these low-energy forms facilitates candidate selection based primarily on efficacy considerations with minimal regard to future complications due to changes to the bulk form. Solubility problems can arise later in development when the drug substance synthetic process is scaled and a highly crystalline, insoluble form is isolated. Compounds with high crystal lattice energy can pose significant solubility problems that cannot be addressed with technologies designed to overcome lipophilicity issues. We estimate that between 10% and 30% of hits identified in high throughput screens could have latent solubility issues associated with crystal packing that would not be predicted based on lipophilicity. Technologies, such as size reduction to nanoparticles (Elan, Skyepharma, Baxter) and stabilization of amorphous forms (SOLIQS), offer options, but these approaches may not always be the answer because of the tendency of some materials to undergo physical changes. Development of alternate systems to address this specific issue is worthy of further investment by DD providers and pharma companies with due consideration of the supply versus demand to avoid development of “excess capacity” and poor adoption of a large number of new technologies.”

Accordingly, substances, compositions, dosage forms and methods that address the above noted problems in the art are needed.

### BRIEF SUMMARY OF THE INVENTION

In an aspect, the invention relates to a substance comprising a complex comprising a drug moiety ionically bound to a counter-ion; wherein a solubility of the complex is greater than a solubility of the drug moiety.

In another aspect, the invention relates to a method of preparing a composition comprising providing a drug moiety; mixing the drug moiety into a solvent that has been titrated to a pH as follows:
(a) for acidic structural elements or acidic residual structural elements, the environmental pH is less than or equal to about the pKa of the drug moiety minus two pH units,
(b) for basic structural elements or basic residual structural elements, the environmental pH is greater than or equal to about the pKa of the drug plus two pH units; adding a counter-ion to the mixture of the drug moiety and the solvent; forming a complex that comprises the drug moiety and the counter-ion; and recovering the complex.

In still another aspect, the invention relates to a method comprising: providing a complex comprising a drug moiety ionically bound to a counter-ion; wherein a solubility of the complex is greater than a solubility of the drug moiety; and administering the complex to a patient.

### BRIEF DESCRIPTION OF THE DRAWINGS

FIG. 1 shows an oral osmotic dosage form useful in the practice of the invention.

FIG. 2 shows an oral osmotic dosage form useful in the practice of the invention.

FIGS. 3A-3C show an oral osmotic dosage form useful in the practice of the invention.

FIG. 4 shows an oral osmotic dosage form useful in the practice of the invention.

FIG. 5 shows an oral osmotic dosage form useful in the practice of the invention.

FIG. 6 shows a synethetic scheme for making a complex according to the invention.

FIG. 7 shows a comparative infra-red spectra.

FIG. 8 shows a comparative X-ray diffraction spectra.

FIG. 9 shows comparative bioavailability data.

FIG. 10 shows comparative dissolution data.

FIG. 11 shows comparative bioavailability data.

### DETAILED DESCRIPTION

### I. DEFINITIONS

All documents cited herein are incorporated by reference in their entirety, for all purposes, as if reproduced herein.

“Complex” means a drug moiety ionically bound to a counter-ion.

“Composition” means one or more of the inventive substances optionally in combination with additional active pharmaceutical ingredients, and optionally in combination with inactive ingredients, such as pharmaceutically-acceptable carriers, excipients, suspension agents, surfactants, disintegrants, binders, diluents, lubricants, stabilizers, antioxidants, osmotic agents, colorants, plasticizers, and the like.

“Counter-ion” means a compound that is capable of forming, or a residue of that compound that has formed, a complex with a drug moiety, wherein the transport moiety serves to interrupt a crystalline structure of the drug moiety, resulting in a more amorphous structure of the complex as compared to the drug moiety. The counter-ion comprises a hydrophobic portion and a(n) acidic, basic, or zwitterionic structural element, or a(n) acidic, basic, or zwitterionic residual structural element. In a preferred embodiment, the pKa of an acidic structural element or acidic residual structural element, including but not limited to zwitterionic structural elements or zwitterionic residual structural elements that comprise acidic structural element or acidic residual structural element, is less than about 7.0, preferably less than about 6.0. In another preferred embodiment, the pKa of a basic structural element or basic residual structural element, including but not limited to zwitterionic structural elements or zwitterionic residual structural elements that comprise basic structural element or basic residual structural element, is greater than about 7.0, preferably greater than about 8.0. These embodiments may be preferred if it is desirable to improve stability of the inventive complex at physiological pH (e.g. pH=about 6.8-7.4). Complexes according to the invention may be formed even the pKa values of the counter-ions do not possess the preferred pKa ranges. Part or all of such complexes may convert back to the uncomplexed drugs after they are solubilized in a neutral pH aqueous solution. Complexing drugs that do not possess preferred pKa ranges may reduce the crystallinity of such drugs and thus increase the dissolution rate and solubility of the drugs in neutral pH aqueous environment before the complexes convert back to the uncomplexed drug. Zwitterionic structural elements or zwitterionic residual structural elements are analyzed in terms of their individual basic structural element or basic residual structural element or their acidic structural element or acidic residual structural element, depending upon how the complex with the drug moiety is to be formed.

In a more preferred embodiment, counter-ions comprise pharmaceutically acceptable acids, including but not limited to carboxylic acids, and salts thereof. In embodiments, counter-ions comprise fatty acids or its salts, benzenesulfonic acid or its salts, benzoic acid or its salts, fumaric acid or its salts, or salicylic acid or its salts. In preferred embodiments the fatty acids or their salts, comprise from 6 to 18 carbon atoms (C₆-C₁₈), more preferably 8 to 16 carbon atoms (C8-C16), even more preferably 10 to 14 carbon atoms (C10-C14), and most preferably 12 carbon atoms (C12).

In more preferred embodiments, counter-ions comprise alkyl sulfates (either saturated or unsaturated) and their salts, such as potassium, magnesium, and sodium salts, including particularly sodium octyl sulfate, sodium decyl sulfate, sodium lauryl sulfate, and sodium tetradecyl sulfate. In preferred embodiments the alkyl sulfate or its salt comprise from 6 to 18 carbon atoms (C6-C18), more preferably 8 to 16 carbon atoms (C8-C16), even more preferably 10 to 14 carbon atoms (C10-C14), and most preferably 12 carbon atoms (C12). Also suitable are other anionic surfactants.

In another more preferred embodiment, counter-ions comprise pharmaceutically acceptable primary amines or salts thereof, particularly primary aliphatic amines (both saturated and unsaturated) or salts thereof, diethanolamine, ethylenediamine, procaine, choline, tromethamine, meglumine, magnesium, aluminum, calcium, zinc, alkyltrimethylammonium hydroxides, alkyltrimethylammonium bromides, benzalkonium chloride and benzethonium chloride. Also useful are other pharmaceutically acceptable compounds that comprise secondary or tertiary amines, and their salts, and cationic surfactants.

“Crystallinity” means the degree to which a chemical entity is present in a crystalline form. This degree can be expressed in units such as percent crystallinity. X-Ray diffraction (“XRD”) techniques can be used to determine crystallinity of substances, including inventive substances. From an XRD spectra, the percent crystallinity may be calculated as:
Percent crystallinity=(sum of net intensity)/(sum of observed intensity)
where the sum of net intensity=integrated area of peak intensity above the baseline, and sum of observed intensity=integrated area of total peak intensity including sum of net intensity and integrated area under baseline. Additional information about use of XRD to determine percent crystallinity may be found in R. Jenkins et al., Introduction to X-Ray Powder Diffractometry, John Wiley & Sonds, Inc. (1996).

“Dosage form” means a pharmaceutical composition in a medium, carrier, vehicle, or device suitable for administration to a patient in need thereof.

“Drug” or “Drug moiety” means a drug, compound, or agent, or a residue of such a drug, compound, or agent that (i) provides some pharmacological effect when administered to a subject, and (ii) possess a solubility in water at neutral pH and at 25 Degrees C. of less than or equal to about 100 micrograms/ml of solution. In a preferable embodiment, the drug possesses a solubility in water at neutral pH and at 25 Degrees C. of less than or equal to about 50 micrograms/ml of solution; more preferably the drug possesses a solubility in water at neutral pH and at 25 Degrees C. of less than or equal to about 10 micrograms/ml of solution. For use in forming a complex, the drug comprises a(n) acidic, basic, or zwitterionic structural element, or a(n) acidic, basic, or zwitterionic residual structural element. In embodiments according to the invention, drug moieties that comprise acidic structural elements or acidic residual structural elements are complexed with counter-ions that comprise basic structural elements or basic residual structural elements. In embodiments according to the invention, drug moieties that comprise basic structural elements or basic residual structural elements are complexed with counter-ions that comprise acidic structural elements or acidic residual structural elements. In embodiments according to the invention, drug moieties that comprise zwitterionic structural elements or zwitterionic residual structural elements are complexed with counter-ions that comprise either acidic or basic structural elements, or acidic or basic residual structural elements. In a preferred embodiment, the pKa of an acidic structural element or acidic residual structural element, including but not limited to zwitterionic structural elements or zwitterionic residual structural elements that comprise acidic structural element or acidic residual structural element, is less than about 7.0, preferably less than about 6.0. In another preferred embodiment, the pKa of a basic structural element or basic residual structural element, including but not limited to zwitterionic structural elements or zwitterionic residual structural elements that comprise basic structural element or basic residual structural element, is greater than about 7.0, preferably greater than about 8.0. These embodiments may be preferred if it is desirable to improve stability of the inventive complex at physiological pH (e.g. pH=about 6.8-7.4). Zwitterionic structural elements or zwitterionic residual structural elements are analyzed in terms of their individual basic structural element or basic residual structural element or their acidic structural element or acidic residual structural element, depending upon how the complex with the counter-ion is to be formed.

“Fatty acid” means any of the group of organic acids of the general formula CH₃(CₙHₓ)COOH where the hydrocarbon chain is either saturated (x=2n, e.g. palmitic acid, CH₃C₁₄H₂₈COOH) or unsaturated (for monounsaturated, x=2n−2, e.g. oleic acid, CH₃C₁₆H₃₀COOH).

“Ionically bound” means a bond or bonding between two atoms that is ionic in nature.

“Patient” means an animal, preferably a mammal, more preferably a human, in need of therapeutic intervention.

“Pharmaceutical composition” means a composition suitable for administration to a patient in need thereof.

“Physical mixture” means a mixture of chemical entities that is heterogeneous at molecular distances, but appears homogeneous at distances of 1 micron or larger. Physical mixtures can be made by conventional blending, or milling techniques. Physical mixtures are distinguishable from the inventive complexes, which produce structural changes in a substance at the molecular level. Physical mixtures that comprise a crystalline material, for instance, will comprise domains of the crystalline material in its crystalline state, separated by domains of the other materials in the physical mixture.

“Residual structural element” means a structural element that is modified by interaction or reaction with another compound, chemical group, ion, atom, or the like. For example, a carboxyl structural element (COOH) interacts with sodium to form a sodium-carboxylate salt, the COO— being a residual structural element.

“Solubility” means the solubility of a species.

“Structural element” means a chemical group that (i) is part of a larger molecule, and (ii) possesses distinguishable chemical functionality. For example, an acidic group or a basic group on a compound is a structural element.

“Substance” means a chemical entity having specific characteristics. A substance according to the invention comprises an inventive complex.

### II. DISCUSSION OF MECHANISM, RESULTS, AND METHODS OF MAKING COMPLEXES

As noted above in Horspool, “[c]ompounds with high crystal lattice energy can pose significant solubility problems that cannot be addressed with technologies designed to overcome lipophilicity issues.” What is needed is approaches that can reduce the crystallinity of drugs, and therefore increase their solubility.

The inventors have unexpectedly discovered that it is possible to provide complexes that comprise a drug moiety having low solubility ionically bound to a counter-ion; wherein a solubility of the complex is greater than a solubility of the drug moiety. In embodiments, the crystallinity of the complex is less than the crystallinity of the drug moiety. Taken together, the invention addresses the needs of the art as described above. The nature of the invention now will be described in more detail.

While not wishing to be limited to the following explanation of possible mechanisms, the inventors reason as follows: Solubility of a substance can be considered to depend, in part, on the attractive forces between substance molecules as compared to the attractive forces between a substance molecule and solvent molecules. Highly crystalline substances may be characterized as having very strong attractive forces between substance molecules as compared to relatively weaker attractive forces between substance molecules and solvent molecules. Thus, highly crystalline substances tend to be less soluble than more amorphous substances.

Researchers have responded by tying to make formulations with less crystalline characteristics. For instance, physical mixtures of crystalline substances with relatively amorphous materials have been proposed, including micronization of the crystalline substance followed by mixing with an amorphous substance. The results have not been completely satisfactory. The inventors hypothesize that this may be the case, in part, because in the microscopic domains of crystalline substances formed by physical mixtures, even the micron or sub-micron sized particles formed using micronization techniques, attractive forces between the substance molecules present in the domain significantly exceed attractive forces between the substance molecule and solvent molecules. This therefore limits transport of the substance from the domain into solution, even with the increased surface area resulting from micronization.

A next step in trying to improve solubility is to produce solid dispersions of crystalline substances. While this technique does reduce the impact of crystalline domains by reducing crystal size (thus increasing surface area available for transport of substance molecules into solution), the resulting dispersions can be unstable at higher loadings due to the tendency of nearby substance molecules to aggregate into crystals. Lower loadings of solid solutions are more stable but increase formulation volume to undesirably high levels.

The inventive complexes address these problems presumably by providing molecular level dispersions at much higher loadings than solid dispersion. The inventive complexes separate substance molecules and are presumed to maintain that separation at a molecular level. This molecular separation can provide improved solubility compared to the crystalline substance by itself or physical mixtures of the crystalline substance with a more amorphous material. The inventive complexes may represent an improvement over solid dispersion because of higher loading levels and also the potential for improved stability once the molecule is in solution.

Complexes according to the invention may be made according to the following general guidelines.

First, the pKa of the drug moiety is established, using literature data or deriving the value using conventional experimental techniques. For drug moieties that comprise only an acidic structural element or an acidic residual structural element, or drug moieties that comprise only a basic structural element or a basic residual structural element, this step can be completed by referencing literature data or generating pKa experimentally using conventional techniques. If the structural element is a zwitterion, or a zwitterionic residue, the next step is to determine whether the acidic or basic group will be the group that forms the complex with the counter-ion. The group that will not be forming the complex by bonding with the transport moiety may be blocked. A preferred method for blocking the non-bonding structural element or residual structural element of a zwitterionic structural element or residual structural element is to adjust the environmental pH so that the non-bonding structural element is not ionized. For instance, to block an acidic structural element, the environmental pH is lowered so that the acidic structural element is not ionized, but the basic structural element is. For blocking of basic structural elements, the pH is raised so that the basic structural element is not ionized but the acidic structural element is. Once the desired structural element has been blocked, the drug moiety may be processed according to the procedure set forth herein.

The drug moiety is then mixed into a solvent that has been titrated to a pH that satisfies the following equation:

For acidic structural elements or acidic residual structural elements:
pH(environment)<=about(pKa (drug)−2) (Equation 1)
In word form, the environmental pH is less than or equal to about the pKa of the drug minus two pH units.

For basic structural elements or basic residual structural elements:
pH(environment)>=about(pKa (drug)+2) (Equation 2)
In word form, the environmental pH is greater than or equal to about the pKa of the drug plus two pH units.

In an embodiment, the drug must dissolve in the solvent at the environmental pH. A preferred solvent is an aqueous solvent, including mixtures of non-aqueous solvents with water.

Next, the counter-ion is added to the solution. In an embodiment, the counter-ion must be able to dissolve in the solution at the environmental pH. Generally, the counter-ion is added in molar excess to the drug moiety.

In an embodiment, the inventive complex precipitates out from solution upon formation. In certain embodiments, while the individual ions are soluble in the solvent, the inventive complexes tend not to be. The precipitate is then recovered using standard techniques (washing, drying, etc.), and then may be used according to the teachings of the present invention.

In other embodiments, the counter-ion is dissolved first in the solvent, and then the drug moiety is added to the solution. The inventive complex is again recovered as a precipitate. In such embodiments, both the drug moiety and the counter-ion must be capable of being dissolved in the solvent at the environmental pH, which is set according to Equations 1 or 2. The counter-ion may still be present in molar excess.

For certain drugs, such as water insoluble drugs, ionization may not be possible using pH titration. In such embodiments, the complex may be formed using organic solvents. The first step is to obtain the drug in its non-ionized (i.e. basic or acidic) form. The drug may then be dissolved in a suitable organic solvent. The counter-ion is then dissolved in the same solvent, preferably in a separate vessel. The counter-ion must also be in its non-ionized (i.e. basic or acidic) form. The counter-ion must be selected such that the difference in pKa between the counter-ion and the drug is at least about 2 pKa units. The drug solution and the counter-ion solution are then mixed and the complex is recovered as a precipitate.

### III. INVENTIVE DOSAGE FORMS

Substances comprising complexes according to the invention may be administered to patients in need thereof. In embodiments, the inventive substances are formulated into dosage forms administerable to patients in need thereof. In preferable embodiments, the substances are formulated into compositions, more preferably pharmaceutical compositions, of which the dosage forms are comprised.

The complexes described herein provide an enhanced solubility, and therefore improved absorption. Dosage forms and methods of treatment using the complex and its enhanced solubility will now be described. It will be appreciated that the dosage forms described below are merely exemplary.

A variety of dosage forms are suitable for use with the inventive complexes. The dosage form may be configured and formulated according to any design that delivers a desired dose of the drug moiety. In certain embodiments, the dosage form is orally administrable and is sized and shaped as a conventional tablet or capsule. Orally administrable dosage forms may be manufactured according to one of various different approaches. For example, the dosage form may be manufactured as a diffusion system, such as a reservoir device or matrix device, a dissolution system, such as encapsulated dissolution systems (including, for example, “tiny time pills”, and beads) and matrix dissolution systems, and combination diffusion/dissolution systems and ion-exchange resin systems, as described in Remington's Pharmaceutical Sciences, 18th Ed., pp. 1682-1685 (1990).

Dosage forms suitable for use in the practice of this invention comprise immediate release (IR), thrice daily (tid) twice daily (bid), and once daily (qd).

One important consideration in the practice of this invention is the physical state of the complex to be delivered by the dosage form. In certain embodiments, the substances according to the invention that comprise inventive complexes may possess a lower melting point than the drug itself, and therefore be in a paste or liquid state. In such cases solid dosage forms may not be suitable for use in the practice of this invention. Instead, dosage forms capable of delivering substances in a paste or liquid state should be used. Alternatively, in certain embodiments, a different counter-ion may be used to raise the melting point of the substances, thus making it more likely that the inventive complexes will be present in a solid form.

A specific example of a dosage form suitable for use with the present invention is an osmotic dosage form. Osmotic dosage forms, in general, utilize osmotic pressure to generate a driving force for imbibing fluid into a compartment formed, at least in part, by a semipermeable wall that permits free diffusion of fluid but not drug or osmotic agent(s), if present. An advantage to osmotic systems is that their operation is pH-independent and, thus, continues at the osmotically determined rate throughout an extended time period even as the dosage form transits the gastrointestinal tract and encounters differing microenvironments having significantly different pH values. A review of such dosage forms is found in Santus and Baker, “Osmotic drug delivery: a review of the patent literature,” Journal of Controlled Release, 35:1-21 (1995). Osmotic dosage forms are also described in detail in the following U.S. patents: U.S. Pat. Nos. 3,845,770; 3,916,899; 3,995,631; 4,008,719; 4,111,202; 4,160,020; 4,327,725; 4,519,801; 4,578,075; 4,681,583; 5,019,397; and 5,156,850.

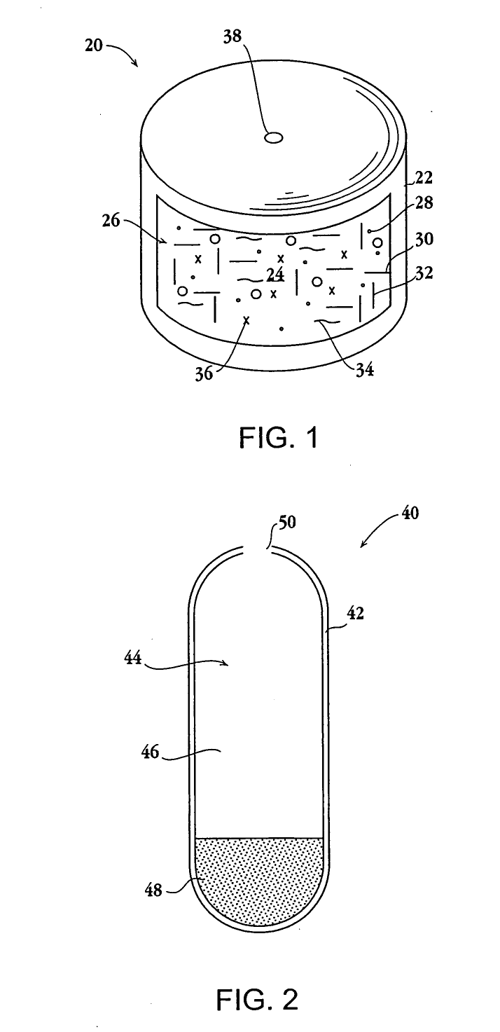

An exemplary dosage form, referred to in the art as an elementary osmotic pump dosage form, is shown in FIG. 1. Dosage form 20, shown in a cutaway view, is also referred to as an elementary osmotic pump, and is comprised of a semi-permeable wall 22 that surrounds and encloses an internal compartment 24. The internal compartment contains a single component layer referred to herein as a drug layer 26, comprising an inventive substance 28 in an admixture with selected excipients. The excipients are adapted to provide an osmotic activity gradient for attracting fluid from an external environment through wall 22 and for forming a deliverable complex formulation upon imbibition of fluid. The excipients may include a suitable suspending agent, also referred to herein as drug carrier 30, a binder 32, a lubricant 34, and an osmotically active agent referred to as an osmagent 36. Exemplary materials useful for these components can be found disclosed throughout the present application.

Semi-permeable wall 22 of the osmotic dosage form is permeable to the passage of an external fluid, such as water and biological fluids, but is substantially impermeable to the passage of components in the internal compartment. Materials useful for forming the wall are essentially nonerodible and are substantially insoluble in biological fluids during the life of the dosage form. Representative polymers for forming the semi-permeable wall include homopolymers and copolymers, such as, cellulose esters, cellulose ethers, and cellulose ester-ethers. Flux-regulating agents can be admixed with the wall-forming material to modulate the fluid permeability of the wall. For example, agents that produce a marked increase in permeability to fluid such as water are often essentially hydrophilic, while those that produce a marked permeability decrease to water are essentially hydrophobic. Exemplary flux regulating agents include polyhydric alcohols, polyalkylene glycols, polyalkylenediols, polyesters of alkylene glycols, and the like.

In operation, the osmotic gradient across wall 22 due to the presence of osmotically-active agents causes gastric fluid to be imbibed through the wall, swelling of the drug layer, and formation of a deliverable complex formulation (e.g., a solution, suspension, slurry or other flowable composition) within the internal compartment. The deliverable inventive substance formulation is released through an exit 38 as fluid continues to enter the internal compartment. Even as drug formulation is released from the dosage form, fluid continues to be drawn into the internal compartment, thereby driving continued release. In this manner, the inventive substance is released in a sustained and continuous manner over an extended time period.

FIG. 2 is a schematic illustration of another exemplary osmotic dosage form. Dosage forms of this type are described in detail in U.S. Pat. Nos. 4,612,008; 5,082,668; and 5,091,190. In brief, dosage form 40, shown in cross-section, has a semi-permeable wall 42 defining an internal compartment 44. Internal compartment 44 contains a bilayered-compressed core having a drug layer 46 and a push layer 48. As will be described below, push layer 48 is a displacement composition that is positioned within the dosage form such that as the push layer expands during use, the materials forming the drug layer are expelled from the dosage form via one or more exit ports, such as exit port 50. The push layer can be positioned in contacting layered arrangement with the drug layer, as illustrated in FIG. 2, or can have one or more intervening layers separating the push layer and drug layer.

Drug layer 46 comprises an inventive substance an admixture with selected excipients, such as those discussed above with reference to FIG. 1 or discussed elsewhere herein. An exemplary dosage form can have a drug layer comprised of an inventive substance, a poly(ethylene oxide) as a carrier, sodium chloride as an osmagent, hydroxypropylmethylcellulose as a binder, and magnesium stearate as a lubricant.

Push layer 48 comprises osmotically active component(s), such as one or more polymers that imbibes an aqueous or biological fluid and swells, that are referred to in the art as an osmopolymer. Osmopolymers are swellable, hydrophilic polymers that interact with water and aqueous biological fluids and swell or expand to a high degree, typically exhibiting a 2-50 fold volume increase. The osmopolymer can be non-crosslinked or crosslinked, and in a preferred embodiment the osmopolymer is at least lightly crosslinked to create a polymer network that is too large and entangled to easily exit the dosage form during use. Examples of polymers that may be used as osmopolymers are provided in the references noted above that describe osmotic dosage forms in detail. A typical osmopolymer is a poly(alkylene oxide), such as poly(ethylene oxide), and a poly(alkali carboxymethylcellulose), where the alkali is sodium, potassium, or lithium. Additional excipients such as a binder, a lubricant, an antioxidant, and a colorant may also be included in the push layer. In use, as fluid is imbibed across the semi-permeable wall, the osmopolymer(s) swell and push against the drug layer to cause release of the drug from the dosage form via the exit port(s).

The push layer can also include a component referred to as a binder, which is typically a cellulose or vinyl polymer, such as poly-n-vinylamide, poly-n-vinylacetamide, poly(vinyl pyrrolidone), poly-n-vinylcaprolactone, poly-n-vinyl-5-methyl-2-pyrrolidone, and the like. The push layer can also include a lubricant, such as sodium stearate or magnesium stearate, and an antioxidant to inhibit the oxidation of ingredients. Representative antioxidants include, but are not limited to, ascorbic acid, ascorbyl palmitate, butylated hydroxyanisole, a mixture of 2 and 3 tertiary-butyl-4-hydroxyanisole, and butylated hydroxytoluene.

An osmagent may also be incorporated into the drug layer and/or the push layer of the osmotic dosage form. Presence of the osmagent establishes an osmotic activity gradient across the semi-permeable wall. Exemplary osmagents include salts, such as sodium chloride, potassium chloride, lithium chloride, etc. and sugars, such as raffinose, sucrose, glucose, lactose, and carbohydrates.

With reference to FIGS. 1-3, the dosage form can optionally include an overcoat (not shown) for color coding the dosage forms according to dose or for providing an immediate release of the inventive complex or another drug.

In use, water flows across the wall and into the push layer and the drug layer. The push layer imbibes fluid and begins to swell and, consequently, pushes on drug layer 44 causing the material in the layer to be expelled through the exit orifice and into the gastrointestinal tract. Push layer 48 is designed to imbibe fluid and continue swelling, thus continually expelling the inventive substance from the drug layer.

In general, release of drug formulation from the inventive oral osmotic dosage form begins after contact with an aqueous environment. For instance, in the dosage form illustrated in FIG. 2, release of the inventive substance, present in the layer adjacent the exit orifice, is released after contact with an aqueous environment and continues for the lifetime of the device.

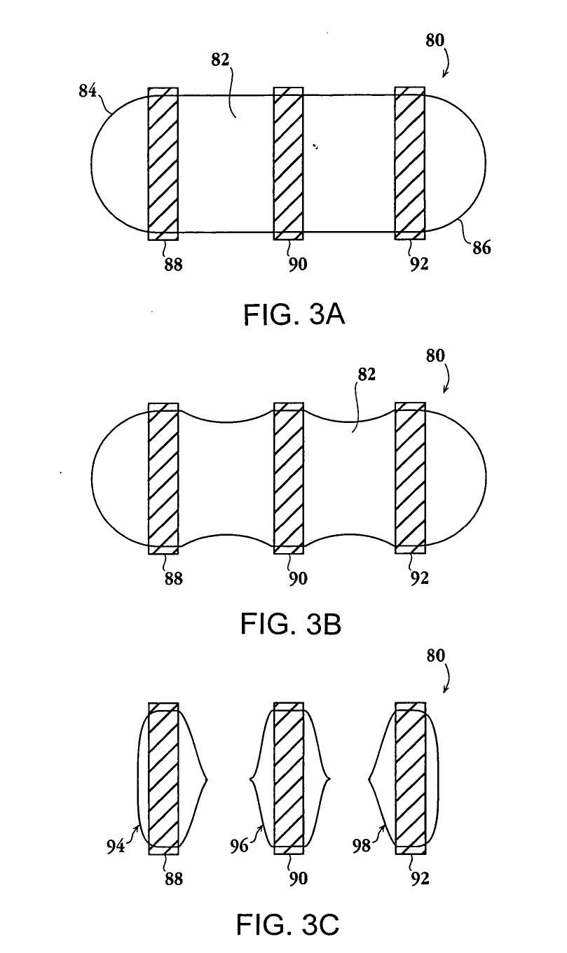

FIGS. 3A-3C illustrate another exemplary dosage form, known in the art and described in U.S. Pat. Nos. 5,534,263; 5,667,804; and 6,020,000. Briefly, a cross-sectional view of a dosage form 80 is shown prior to ingestion into the gastrointestinal tract in FIG. 3A. The dosage form is comprised of a cylindrically shaped matrix 82 comprising an inventive substance. Ends 84, 86 of matrix 82 are preferably rounded and convex in shape in order to ensure ease of ingestion. Bands 88, 90, and 92 concentrically surround the cylindrical matrix and are formed of a material that is relatively insoluble in an aqueous environment. Suitable materials are set forth in the patents noted above and elsewhere herein.

After ingestion of dosage form 80, regions of matrix 82 between bands 88, 90, 92 begin to erode, as illustrated in FIG. 3B. Erosion of the matrix initiates release of the inventive substance into the fluidic environment of the G.I. tract. As the dosage form continues transit through the G.I. tract, the matrix continues to erode, as illustrated in FIG. 3C. Here, erosion of the matrix has progressed to such an extent that the dosage form breaks into three pieces, 94, 96, 98. Erosion will continue until the matrix portions of each of the pieces have completely eroded. Bands 94, 96, 98 will thereafter be expelled from the G.I. tract.

The present invention provides a liquid formulation of substances for use with oral osmotic devices. Oral osmotic devices for delivering liquid formulations and methods of using them are known in the art, for example, as described and claimed in the following U.S. patents owned by ALZA corporation: U.S. Pat. Nos. 6,419,952; 6,174,547; 6,551,613; 5,324,280; 4,111,201; and 6,174,547. Methods of using oral osmotic devices for delivering therapeutic agents at an ascending rate of release can be found in International Application Numbers WO 98/06380, WO 98/23263, and WO 99/62496.

Exemplary liquid carriers for the present invention include lipophilic solvents (e.g., oils and lipids), surfactants, and hydrophilic solvents. Exemplary lipophilic solvents, for example, include, but are not limited to, Capmul PG-8, Caprol MPGO, Capryol 90, Plurol Oleique CC 497, Capmul MCM, Labrafac PG, N-Decyl Alcohol, Caprol 10G100, Oleic Acid, Vitamin E, Maisine 35-1, Gelucire 33/01, Gelucire 44/14, Lauryl Alcohol, Captex 355EP, Captex 500, Capylic/Caplic Triglyceride, Peceol, Caprol ET, Labrafil M2125 CS, Labrafac CC, Labrafil M 1944 CS, Captex 8277, Myvacet 9-45, Isopropyl Nyristate, Caprol PGE 860, Olive Oil, Plurol Oleique, Peanut Oil, Captex 300 Low C6, and Capric Acid. Exemplary surfactants for example, include, but are not limited to, Vitamin E TPGS, Cremophor EL-P, Labrasol, Tween 20, Cremophor RH40, Pluronic L-121, Acconon S-35, Pluronic L-31, Pluronic L-35, Pluronic L-44, Tween 80, Pluronic L-64, Solutol HS-15, Span 20, Cremophor EL, Span 80, Pluronic L-43, and Tween 60. Exemplary hydrophilic solvents for example, include, but are not limited to, Isosorbide Dimethyl Ether, Polyethylene Glycol 400 (PEG-3000), Transcutol HP, Polyethylene Glycol 400 (PEG4000), Polyethylene Glycol 400 (PEG-300), Polyethylene Glycol 400 (PEG-6000), Polyethylene Glycol 400 (PEG400), Polyethylene Glycol 400 (PEG-8000), Polyethylene Glycol 400 (PEG-600), and Propylene Glycol (PG).

In one embodiment, a liquid formulation comprises from about 10% to about 90% of a substance according to the invention, and about 10% to about 90% of one or more liquid carriers. For example, in some embodiments, the liquid formulation will comprise a substance according to the invention and a hydrophilic solvent such as PG. In such embodiments, the liquid formulation can comprise from about 10% to about 90% of an inventive substances and about 10% to about 90% of the hydrophilic solvent. In one preferred embodiment, the liquid carrier can comprise about 50% surfactant, such as Cremophor EL, solutol, or Tween 80, and about 50% hydrophilic solvent, such as PG.

The skilled practitioner will understand that any formulation comprising a sufficient dosage of inventive substances solubilized in a liquid carrier suitable for administration to a subject and for use in an osmotic device can be used in the present invention. In one exemplary embodiment of the present invention, the liquid carrier is PG, Solutol, Cremophor EL, or a combination thereof.

The liquid formulation according to the present invention can also comprise, for example, additional excipients such as an antioxidant, permeation enhancer and the like. Antioxidants can be provided to slow or effectively stop the rate of any autoxidizable material present in the capsule. Representative antioxidants can comprise a member selected from the group of ascorbic acid; alpha tocopherol; ascorbyl palmitate; ascorbates; isoascorbates; butylated hydroxyanisole; butylated hydroxytoluene; nordihydroguiaretic acid; esters of garlic acid comprising at least 3 carbon atoms comprising a member selected from the group consisting of propyl gallate, octyl gallate, decyl gallate, decyl gallate; 6-ethoxy-2,2,4-trimethyl-1,2-dihydro-guinoline; N-acetyl-2,6-di-t-butyl-p-aminophenol; butyl tyrosine; 3-tertiarybutyl-4-hydroxyanisole; 2-tertiary-butyl-4-hydroxyanisole; 4-chloro-2,6-ditertiary butyl phenol; 2,6-ditertiary butyl p-methoxy phenol; 2,6-ditertiary butyl-p-cresol: polymeric antioxidants; trihydroxybutyro-phenone physiologically acceptable salts of ascorbic acid, erythorbic acid, and ascorbyl acetate; calcium ascorbate; sodium ascorbate; sodium bisulfite; and the like. The amount of antioxidant used for the present purposes, for example, can be about 0.001% to 25% of the total weight of the composition present in the lumen. Antioxidants are known to the prior art in U.S. Pat. Nos. 2,707,154; 3,573,936; 3,637,772; 4,038,434; 4,186,465 and 4,559,237, each of which is hereby incorporated by reference in its entirety for all purposes.

The inventive liquid formulation can comprise permeation enhancers that facilitate absorption of the drug in the environment of use. Such enhancers can, for example, open the so-called “tight junctions” in the gastrointestinal tract or modify the effect of cellular components, such a p-glycoprotein and the like. Suitable enhancers can include alkali metal salts of salicyclic acid, such as sodium salicylate, caprylic or capric acid, such as sodium caprylate or sodium caprate, and the like. Enhancers can include, for example, the bile salts, such as sodium deoxycholate. Various p-glycoprotein modulators are described in U.S. Pat. Nos. 5,112,817 and 5,643,909. Various other absorption enhancing compounds and materials are described in U.S. Pat. No. 5,824,638. Enhancers can be used either alone or as mixtures in combination with other enhancers.

In certain embodiments, the inventive substances are administered as a self-emulsifying formulation. Like the other liquid carriers, the surfactant functions to prevent aggregation, reduce interfacial tension between constituents, enhance the free-flow of constituents, and lessen the incidence of constituent retention in the dosage form. The emulsion formulation of this invention comprises a surfactant that imparts emulsification. Exemplary surfactants can also include, for example, in addition to the surfactants listed above, a member selected from the group consisting of polyoxyethylenated castor oil comprising 9 moles of ethylene oxide, polyoxyethylenated castor oil comprising 15 moles of ethylene oxide, polyoxyethylene caster oil comprising 20 moles of ethylene oxide, polyoxyethylenated caster oil comprising 25 moles of ethylene oxide, polyoxyethylenated caster oil comprising 40 moles of ethylene oxide, polyoxyethylenated castor oil comprising 52 moles of ethylene oxide, polyoxyethylenated sorbitan monopalmitate comprising 20 moles of ethylene oxide, polyoxyethylenated sorbitan monostearate comprising 20 moles of ethylene oxide, polyoxyethylenated sorbitan monostearate comprising 4 moles of ethylene oxide, polyoxyethylenated sorbitan tristearate comprising 20 moles of ethylene oxide, polyoxyethylenated sorbitan monostearate comprising 20 moles of ethylene oxide, polyoxyethylenated sorbitan trioleate comprising 20 moles of ethylene oxide, polyoxyethylene lauryl ether, polyoxyethylenated stearic acid comprising 40 moles of ethylene oxide, polyoxyethylenated stearic acid comprising 50 moles of ethylene oxide, polyoxyethylenated stearyl alcohol comprising 2 moles of ethylene oxide, and polyoxyethylenated oleyl alcohol comprising 2 moles of ethylene oxide. The surfactants are available from Atlas Chemical Industries.

The drug emulsified formulations of the present invention can initially comprise an oil and a non-ionic surfactant. The oil phase of the emulsion comprises any pharmaceutically acceptable oil which is not immiscible with water. The oil can be an edible liquid such as a non-polar ester of an unsaturated fatty acid, derivatives of such esters, or mixtures of such esters. The oil can be vegetable, mineral, animal or marine in origin. Examples of non-toxic oils can also include, for example, in addition to the surfactants listed above, a member selected from the group consisting of peanut oil, cottonseed oil, sesame oil, corn oil, almond oil, mineral oil, castor oil, coconut oil, palm oil, cocoa butter, safflower, a mixture of mono- and diglycerides of 16 to 18 carbon atoms, unsaturated fatty acids, fractionated triglycerides derived from coconut oil, fractionated liquid triglycerides derived from short chain 10 to 15 carbon atoms fatty acids, acetylated monoglycerides, acetylated diglycerides, acetylated triglycerides, olein known also as glyceral trioleate, palmitin known as glyceryl tripalmitate, stearin known also as glyceryl tristearate, lauric acid hexylester, oleic acid oleylester, glycolyzed ethoxylated glycerides of natural oils, branched fatty acids with 13 molecules of ethyleneoxide, and oleic acid decylester. The concentration of oil, or oil derivative in the emulsion formulation can be from about 1 wt % to about 40 wt %, with the wt % of all constituents in the emulsion preparation equal to 100 wt %. The oils are disclosed in Pharmaceutical Sciences by Remington, 17th Ed., pp. 403-405, (1985) published by Mark Publishing Co., in Encyclopedia of Chemistry, by Van Nostrand Reinhold, 4th Ed., pp. 644-645, (1984) published by Van Nostrand Reinhold Co.; and in U.S. Pat. No. 4,259,323.

The amount of inventive substances incorporated in the dosage forms of the present invention is generally from about 10% to about 90% by weight of the composition depending upon the therapeutic indication and the desired administration period, e.g., every 12 hours, every 24 hours, and the like. Depending on the dose of drug desired to be administered, one or more of the dosage forms can be administered.

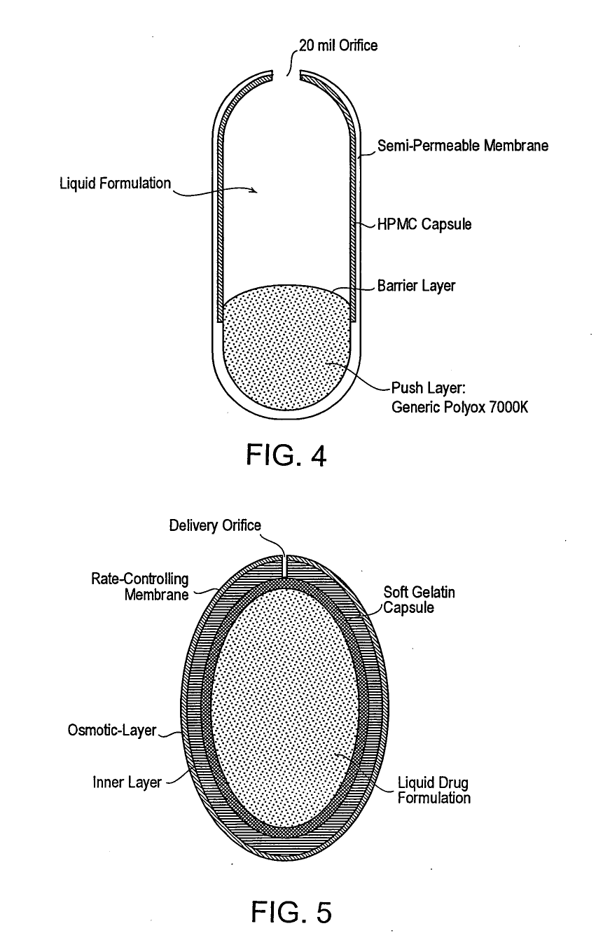

The osmotic dosage forms of the present invention can possess two distinct forms, a soft capsule form (shown in FIG. 4) and a hard capsule form (shown in FIG. 5). The soft capsule, as used by the present invention, preferably in its final form comprises one piece. The one-piece capsule is of a sealed construction encapsulating the drug formulation therein. The capsule can be made by various processes including the plate process, the rotary die process, the reciprocating die process, and the continuous process. An example of the plate process is as follows. The plate process uses a set of molds. A warm sheet of a prepared capsule lamina-forming material is laid over the lower mold and the formulation poured on it. A second sheet of the lamina-forming material is placed over the formulation followed by the top mold. The mold set is placed under a press and a pressure applied, with or without heat, to form a unit capsule. The capsules are washed with a solvent for removing excess agent formulation from the exterior of the capsule, and the air-dried capsule is encapsulated with a semipermeable wall. The rotary die process uses two continuous films of capsule lamina-forming material that are brought into convergence between a pair of revolving dies and an injector wedge. The process fills and seals the capsule in dual and coincident operations. In this process, the sheets of capsule lamina-forming material are fed over guide rolls, and then down between the wedge injector and the die rolls. The agent formulation to be encapsulated flows by gravity into a positive displacement pump. The pump meters the agent formulation through the wedge injector and into the sheets between the die rolls. The bottom of the wedge contains small orifices lined up with the die pockets of the die rolls. The capsule is about half-sealed when the pressure of pumped agent formulation forces the sheets into the die pockets, wherein the capsules are simultaneously filled, shaped, hermetically sealed and cut from the sheets of lamina-forming materials. The sealing of the capsule is achieved by mechanical pressure on the die rolls and by heating of the sheets of lamina-forming materials by the wedge. After manufacture, the agent formulation-filled capsules are dried in the presence of forced air, and a semipermeable lamina encapsulated thereto.

The reciprocating die process produces capsules by leading two films of capsule lamina-forming material between a set of vertical dies. The dies as they close, open, and close perform as a continuous vertical plate forming row after row of pockets across the film. The pockets are filled with an inventive formulation, and as the pockets move through the dies, they are sealed, shaped, and cut from the moving film as capsules filled with agent formulation. A semipermeable encapsulating lamina is coated thereon to yield the capsule. The continuous process is a manufacturing system that also uses rotary dies, with the added feature that the process can successfully fill active agent in dry powder form into a soft capsule, in addition to encapsulating liquids. The filled capsule of the continuous process is encapsulated with a semipermeable polymeric material to yield the capsule. Procedures for manufacturing soft capsules are disclosed in U.S. Pat. No. 4,627,850 and U.S. Pat. No. 6,419,952.

The dosage forms of the present invention can also be made from an injection-moldable composition by an injection-molding technique. Injection-moldable compositions provided for injection-molding into the semipermeable wall comprise a thermoplastic polymer, or the compositions comprise a mixture of thermoplastic polymers and optional injection-molding ingredients. The thermoplastic polymer that can be used for the present purpose comprise polymers that have a low softening point, for example, below 200° C., preferably within the range of 40° C. to 180° C. The polymers, are preferably synthetic resins, addition polymerized resins, such as polyamides, resins obtained from diepoxides and primary alkanolamines, resins of glycerine and phthalic anhydrides, polymethane, polyvinyl resins, polymer resins with end-positions free or esterified carboxyl or caboxamide groups, for example with acrylic acid, acrylic amide, or acrylic acid esters, polycaprolactone, and its copolymers with dilactide, diglycolide, valerolactone and decalactone, a resin composition comprising polycaprolactone and polyalkylene oxide, and a resin composition comprising polycaprolactone, a polyalkylene oxide such as polyethylene oxide, poly(cellulose) such as poly(hydroxypropylmethylcellulose), poly(hydroxyethylmethylcellulose), and poly(hydroxypropylcellulose). The membrane forming composition can comprise optional membrane-forming ingredients such as polyethylene glycol, talcum, polyvinylalcohol, lactose, or polyvinyl pyrrolidone. The compositions for forming an injection-molding polymer composition can comprise 100% thermoplastic polymer. The composition in another embodiment comprises 10% to 99% of a thermoplastic polymer and 1% to 90% of a different polymer with the total equal to 100%. The invention provides also a thermoplastic polymer composition comprising 1% to 98% of a first thermoplastic polymer, 1% to 90% of a different, second polymer and 1% to 90% of a different, third polymer with all polymers equal to 100%. Representation composition comprises 20% to 90% of thermoplastic polycaprolactone and 10% to 80% of poly(alkylene oxide); a composition comprising 20% to 90% polycaprolactone and 10% to 60% of poly(ethylene oxide) with the ingredients equal to 100%; a composition comprising 10% to 97% of polycaprolactone, 10% to 97% poly(alkylene oxide), and 1% to 97% of poly(ethylene glycol) with all ingredients equal to 100%; a composition comprising 20% to 90% polycaprolactone and 10% to 80% of poly(hydroxypropylcellulose) with all ingredients equal to 100%; and a composition comprising 1% to 90% polycaprolactone, 1% to 90% poly(ethylene oxide), 1% to 90% poly(hydroxypropylcellulose) and 1% to 90% poly(ethylene glycol) with all ingredients equal to 100%. The percent expressed is weight percent wt %.

In another embodiment of the invention, a composition for injection-molding to provide a membrane can be prepared by blending a composition comprising a polycaprolactone 63 wt %, polyethylene oxide 27 wt %, and polyethylene glycol 10 wt % in a conventional mixing machine, such as a Moriyama™ Mixer at 65° C. to 95° C., with the ingredients added to the mixer in the following addition sequence, polycaprolactone, polyethylene oxide and polyethylene glycol. In one example, all the ingredients are mixed for 135 minutes at a rotor speed of 10 to 20 rpm. Next, the blend is fed to a Baker Perkins Kneader™ extruder at 80° C. to 90° C., at a pump speed of 10 rpm and a screw speed of 22 rpm, and then cooled to 10° C. to 12° C., to reach a uniform temperature. Then, the cooled extruded composition is fed to an Albe Pelletizer, converted into pellets at 250° C., and a length of 5 mm. The pellets next are fed into an injection-molding machine, an Arburg Allrounder™ at 200° F. to 350° C. (93° C. to 177° C.), heated to a molten polymeric composition, and the liquid polymer composition forced into a mold cavity at high pressure and speed until the mold is filled and the composition comprising the polymers are solidified into a preselected shape. The parameters for the injection-molding consists of a band temperature through zone 1 to zone 5 of the barrel of 195° F. (91° C.) to 375° F., (191° C.), an injection-molding pressure of 1818 bar, a speed of 55 cm3/s, and a mold temperature of 75° C. The injection-molding compositions and injection-molding procedures are disclosed in U.S. Pat. No. 5,614,578.

Alternatively, the capsule can be made conveniently in two parts, with one part (the “cap”) slipping over and capping the other part (the “body”) as long as the capsule is deformable under the forces exerted by the expandable layer and seals to prevent leakage of the liquid, active agent formulation from between the telescoping portions of the body and cap. The two parts completely surround and capsulate the internal lumen that contains the liquid, active agent formulation, which can contain useful additives. The two parts can be fitted together after the body is filled with a preselected formulation. The assembly can be done by slipping or telescoping the cap section over the body section, and sealing the cap and body, thereby completely surrounding and encapsulating the formulation of active agent.

Soft capsules typically have a wall thickness that is greater than the wall thickness of hard capsules. For example, soft capsules can, for example, have a wall thickness on the order of 10-40 mils, about 20 mils being typical, whereas hard capsules can, for example, have a wall thickness on the order of 2-6 mils, about 4 mils being typical.

In one embodiment of the dosage system, a soft capsule can be of single unit construction and can be surrounded by an unsymmetrical hydro-activated layer as the expandable layer. The expandable layer will generally be unsymmetrical and have a thicker portion remote from the exit orifice. As the hydro-activated layer imbibes and/or absorbs external fluid, it expands and applies a push pressure against the wall of capsule and optional barrier layer and forces active agent formulation through the exit orifice. The presence of an unsymmetrical layer functions to assure that the maximum dose of agent is delivered from the dosage form, as the thicker section of layer distant from passageway swells and moves towards the orifice.

In yet another configuration, the expandable layer can be formed in discrete sections that do not entirely encompass an optionally barrier layer-coated capsule. The expandable layer can be a single element that is formed to fit the shape of the capsule at the area of contact. The expandable layer can be fabricated conveniently by tableting to form the concave surface that is complementary to the external surface of the barrier-coated capsule. Appropriate tooling such as a convex punch in a conventional tableting press can provide the necessary complementary shape for the expandable layer. In this case, the expandable layer is granulated and compressed, rather than formed as a coating. The methods of formation of an expandable layer by tableting are well known, having been described, for example in U.S. Pat. Nos. 4,915,949; 5,126,142; 5,660,861; 5,633,011; 5,190,765; 5,252,338; 5,620,705; 4,931,285; 5,006,346; 5,024,842; and 5,160,743.

In some embodiments, a barrier layer can be first coated onto the capsule and then the tableted, expandable layer is attached to the barrier-coated capsule with a biologically compatible adhesive. Suitable adhesives include, for example, starch paste, aqueous gelatin solution, aqueous gelatin/glycerin solution, acrylate-vinylacetate based adhesives such as Duro-Tak adhesives (National Starch and Chemical Company), aqueous solutions of water soluble hydrophilic polymers such as hydroxypropyl methyl cellulose, hydroxymethyl cellulose, hydroxyethyl cellulose, and the like. That intermediate dosage form can be then coated with a semipermeable layer. The exit orifice is formed in the side or end of the capsule opposite the expandable layer section. As the expandable layer imbibes fluid, it will swell. Since it is constrained by the semipermeable layer, as it expands it will compress the barrier-coated capsule and express the liquid, active agent formulation from the interior of the capsule into the environment of use.

The hard capsules are typically composed of two parts, a cap and a body, which are fitted together after the larger body is filled with a preselected appropriate formulation. This can be done by slipping or telescoping the cap section over the body section, thus completely surrounding and encapsulating the useful agent formulation. Hard capsules can be made, for example, by dipping stainless steel molds into a bath containing a solution of a capsule lamina-forming material to coat the mold with the material. Then, the molds are withdrawn, cooled, and dried in a current of air. The capsule is stripped from the mold and trimmed to yield a lamina member with an internal lumen. The engaging cap that telescopically caps the formulation receiving body is made in a similar manner. Then, the closed and filled capsule can be encapsulated with a semipermeable lamina. The semipermeable lamina can be applied to capsule parts before or after parts and are joined into the final capsule. In another embodiment, the hard capsules can be made with each part having matched locking rings near their opened end that permit joining and locking together the overlapping cap and body after filling with formulation. In this embodiment, a pair of matched locking rings are formed into the cap portion and the body portion, and these rings provide the locking means for securely holding together the capsule. The capsule can be manually filled with the formulation, or they can be machine filled with the formulation. In the final manufacture, the hard capsule is encapsulated with a semipermeable lamina permeable to the passage of fluid and substantially impermeable to the passage of useful agent. Methods of forming hard cap dosage forms are described in U.S. Pat. No. 6,174,547, U.S. Pat. Nos. 6,596,314, 6,419,952, and 6,174,547.

The hard and soft capsules can comprise, for example, gelatin; gelatin having a viscosity of 15 to 30 millipoises and a bloom strength up to 150 grams; gelatin having a bloom value of 160 to 250; a composition comprising gelatin, glycerine, water and titanium dioxide; a composition comprising gelatin, erythrosin, iron oxide and titanium dioxide; a composition comprising gelatin, glycerine, sorbitol, potassium sorbate and titanium dioxide; a composition comprising gelatin, acacia glycerine, and water; and the like. Materials useful for forming capsule wall are known in U.S. Pat. No. 4,627,850; and in 4,663,148. Alternatively, the capsules can be made out of materials other than gelatin (see for example, products made by BioProgres plc).

The capsules typically can be provided, for example, in sizes from about 3 to about 22 minims (1 minim being equal to 0.0616 ml) and in shapes of oval, oblong or others. They can be provided in standard shape and various standard sizes, conventionally designated as (000), (00), (0), (1), (2), (3), (4), and (5). The largest number corresponds to the smallest size. Non-standard shapes can be used as well. In either case of soft capsule or hard capsule, non-conventional shapes and sizes can be provided if required for a particular application.

The osmotic devices of the present invention comprise a semipermeable wall permeable to the passage of exterior biological fluid and substantially impermeable to the passage of drug formulation. The selectively permeable compositions used for forming the wall are essentially non-erodible and they are insoluble in biological fluids during the life of the osmotic system. The semipermeable wall comprises a composition that does not adversely affect the host, the drug formulation, an osmopolymer, osmagent and the like. Representative polymers for forming semipermeable wall comprise semipermeable homopolymers, semipermeable copolymers, and the like. In one presently preferred embodiment, the compositions can comprise cellulose esters, cellulose ethers, and cellulose ester-ethers. The cellulosic polymers typically have a degree of substitution, “D.S.”, on their anhydroglucose unit from greater than 0 up to 3 inclusive. By degree of substitution is meant the average number of hydroxyl groups originally present on the anhydroglucose unit that are replaced by a substituting group, or converted into another group. The anhydroglucose unit can be partially or completely substituted with groups such as acyl, alkanoyl, alkenoyl, aroyl, alkyl, alkoxy, halogen, carboalkyl, alkylcarbamate, alkylcarbonate, alkylsulfonate, alkylsulfamate, semipermeable polymer forming groups, and the like. The semipermeable compositions typically include a member selected from the group consisting of cellulose acylate, cellulose diacylate, cellulose triacylate, cellulose triacetate, cellulose acetate, cellulose diacetate, cellulose triacetate, mono-, di- and tri-cellulose alkanylates, mono-, di-, and tri-alkenylates, mono-, di-, and tri-aroylates, and the like. Exemplary polymers can include, for example, cellulose acetate have a D.S. of 1.8 to 2.3 and an acetyl content of 32 to 39.9%; cellulose diacetate having a D.S. of 1 to 2 and an acetyl content of 21 to 35%, cellulose triacetate having a D.S. of 2 to 3 and an acetyl content of 34 to 44.8%, and the like. More specific cellulosic polymers include cellulose propionate having a D.S. of 1.8 and a propionyl content of 38.5%; cellulose acetate propionate having an acetyl content of 1.5 to 7% and an acetyl content of 39 to 42%; cellulose acetate propionate having an acetyl content of 2.5 to 3%, an average propionyl content of 39.2 to 45%, and a hydroxyl content of 2.8 to 5.4%; cellulose acetate butyrate having a D.S. of 1.8, an acetyl content of 13 to 15%, and a butyryl content of 34 to 39%; cellulose acetate butyrate having an acetyl content of 2 to 29%, a butyryl content of 17 to 53%, and a hydroxyl content of 0.5 to 4.7%; cellulose triacylates having a D.S. of 2.6 to 3 such as cellulose trivalerate, cellulose trilamate, cellulose tripalmitate, cellulose trioctanoate, and cellulose tripropionate; cellulose diesters having a D.S. of 2.2 to 2.6 such as cellulose disuccinate, cellulose dipalmitate, cellulose dioctanoate, cellulose dicarpylate, and the like; mixed cellulose esters such as cellulose acetate valerate, cellulose acetate succinate, cellulose propionate succinate, cellulose acetate octanoate, cellulose valerate palmitate, cellulose acetate heptonate, and the like. Semipermeable polymers are known in U.S. Pat. No. 4,077,407 and they can be synthesized by procedures described in Encyclopedia of Polymer Science and Technology, Vol. 3, pages 325 to 354, 1964, published by Interscience Publishers, Inc., New York. Additional semipermeable polymers for forming the semipermeable wall can comprise, for example, cellulose acetaldehyde dimethyl acetate; cellulose acetate ethylcarbamate; cellulose acetate methylcarbamate; cellulose dimethylaminoacetate; semipermeable polyamide; semipermeable polyurethanes; semipermeable sulfonated polystyrenes; cross-linked selectively semipermeable polymers formed by the coprecipitation of a polyanion and a polycation as disclosed in U.S. Pat. Nos. 3,173,876; 3,276,586; 3,541,005; 3,541,006; and 3,546,142; semipermeable polymers as disclosed in U.S. Pat. No. 3,133,132; semipermeable polystyrene derivatives; semipermeable poly (sodium styrenesulfonate); semipermeable poly (vinylbenzyltremethylammonium chloride); semipermeable polymers, exhibiting a fluid permeability of 10-5 to 10-2 (cc. mil/cm hr·atm) expressed as per atmosphere of hydrostatic or osmotic pressure differences across a semipermeable wall. The polymers are known to the art in U.S. Pat. Nos. 3,845,770; 3,916,899; and 4,160,020; and in Handbook of Common Polymers, by Scott, J. R., and Roff, W. J., 1971, published by CRC Press, Cleveland. Ohio.

The semipermeable wall can also comprise a flux regulating agent. The flux regulating agent is a compound added to assist in regulating the fluid permeability or flux through the wall. The flux regulating agent can be a flux enhancing agent or a decreasing agent. The agent can be preselected to increase or decrease the liquid flux. Agents that produce a marked increase in permeability to fluids such as water are often essentially hydrophilic, while those that produce a marked decrease to fluids such as water are essentially hydrophobic. The amount of regulator in the wall when incorporated therein generally is from about 0.01% to 20% by weight or more. The flux regulator agents in one embodiment that increase flux include, for example, polyhydric alcohols, polyalkylene glycols, polyalkylenediols, polyesters of alkylene glycols, and the like. Typical flux enhancers include polyethylene glycol 300, 400, 600, 1500, 4000, 6000, poly(ethylene glycol-co-propylene glycol), and the like; low molecular weight gylcols such as polypropylene glycol, polybutylene glycol and polyamylene glycol: the polyalkylenediols such as poly(1,3-propanediol), poly(1,4-butanediol), poly(1,6-hexanediol), and the like; aliphatic diols such as 1,3-butylene glycol, 1,4-pentamethylene glycol, 1,4-hexamethylene glycol, and the like; alkylene triols such as glycerine, 1,2,3-butanetriol, 1,2,4-hexanetriol, 1,3,6-hexanetriol and the like; esters such as ethylene glycol dipropionate, ethylene glycol butyrate, butylene glucol dipropionate, glycerol acetate esters, and the like. Representative flux decreasing agents include, for example, phthalates substituted with an alkyl or alkoxy or with both an alkyl and alkoxy group such as diethyl phthalate, dimethoxyethyl phthalate, dimethyl phthalate, and [di(2-ethylhexyl)phthalate], aryl phthalates such as triphenyl phthalate, and butyl benzyl phthalate; insoluble salts such as calcium sulphate, barium sulphate, calcium phosphate, and the like; insoluble oxides such as titanium oxide; polymers in powder, granule and like form such as polystyrene, polymethylmethacrylate, polycarbonate, and polysulfone; esters such as citric acid esters esterfied with long chain alkyl groups; inert and substantially water impermeable fillers; resins compatible with cellulose based wall forming materials, and the like.

Other materials that can be used to form the semipermeable wall for imparting flexibility and elongation properties to the wall, for making the wall less-to-nonbrittle and to render tear strength, include, for example, phthalate plasticizers such as dibenzyl phthalate, dihexyl phthalate, butyl octyl phthalate, straight chain phthalates of six to eleven carbons, di-isononyl phthalte, di-isodecyl phthalate, and the like. The plasticizers include nonphthalates such as triacetin, dioctyl azelate, epoxidized tallate, tri-isoctyl trimellitate, tri-isononyl trimellitate, sucrose acetate isobutyrate, epoxidized soybean oil, and the like. The amount of plasticizer in a wall when incorporated therein is about 0.01% to 20% weight, or higher.

The semipermeable wall surrounds and forms a compartment containing a plurality of layers, one of which is an expandable layer which in some embodiments, can contain osmotic agents. The expandable layer comprises in one embodiment a hydroactivated composition that swells in the presence of water, such as that present in gastric fluids. Conveniently, it can comprise an osmotic composition comprising an osmotic solute that exhibits an osmotic pressure gradient across the semipermeable layer against an external fluid present in the environment of use. In another embodiment, the hydro-activated layer comprises a hydrogel that imbibes and/or absorbs fluid into the layer through the outer semipermeable wall. The semipermeable wall is non-toxic. It maintains its physical and chemical integrity during operation and it is essentially free of interaction with the expandable layer.

The expandable layer in one preferred embodiment comprises a hydroactive layer comprising a hydrophilic polymer, also known as osmopolymers. The osmopolymers exhibit fluid imbibition properties. The osmopolymers are swellable, hydrophilic polymers, which osmopolymers interact with water and biological aqueous fluids and swell or expand to an equilibrium state. The osmopolymers exhibit the ability to swell in water and biological fluids and retain a significant portion of the imbibed fluid within the polymer structure. The osmopolymers swell or expand to a very high degree, usually exhibiting a 2 to 50 fold volume increase. The osmopolymers can be noncross-linked or cross-linked. The swellable, hydrophilic polymers are in one embodiment lightly cross-linked, such cross-links being formed by covalent or ionic bonds or residue crystalline regions after swelling. The osmopolymers can be of plant, animal or synthetic origin.

The osmopolymers are hydrophilic polymers. Hydrophilic polymers suitable for the present purpose include poly (hydroxy-alkyl methacrylate) having a molecular weight of from 30,000 to 5,000,000; poly (vinylpyrrolidone) having a molecular weight of from 10,000 to 360,000; anionic and cationic hydrogels; polyelectrolytes complexes; poly (vinyl alcohol) having a low acetate residual, cross-linked with glyoxal, formaldehyde, or glutaraldehyde and having a degree of polymerization of from 200 to 30,000; a mixture of methyl cellulose, cross-linked agar and carboxymethyl cellulose; a mixture of hydroxypropyl methylcellulose and sodium carboxymethylcellulose; a mixture of hydroxypropyl ethylcellulose and sodium carboxymethyl cellulose, a mixture of sodium carboxymethylcellulose and methylcellulose, sodium carboxymethylcellulose; potassium carboxymethylcellulose; a water insoluble, water swellable copolymer formed from a dispersion of finely divided copolymer of maleic anhydride with styrene, ethylene, propylene, butylene or isobutylene crosslinked with from 0.001 to about 0.5 moles of saturated cross-linking agent per mole of maleic anhydride per copolymer; water swellable polymers of N-vinyl lactams; polyoxyethylene-polyoxypropylene gel; carob gum; polyacrylic gel; polyester gel; polyuria gel; polyether gel, polyamide gel; polycellulosic gel; polygum gel; initially dry hydrogels that imbibe and absorb water which penetrates the glassy hydrogel and lowers its glass temperature; and the like.

Representative of other osmopolymers can comprise polymers that form hydrogels such as Carbopol™. acidic carboxypolymer, a polymer of acrylic acid cross-linked with a polyallyl sucrose, also known as carboxypolymethylene, and carboxyvinyl polymer having a molecular weight of 250,000 to 4,000,000; Cyanamer™ polyacrylamides; cross-linked water swellable indenemaleic anhydride polymers; Good-rite™ polyacrylic acid having a molecular weight of 80,000 to 200,000; Polyox™ polyethylene oxide polymer having a molecular weight of 100,000 to 5,000,000 and higher; starch graft copolymers; Aqua-Keeps™ acrylate polymer polysaccharides composed of condensed glucose units such as diester cross-linked polygluran; and the like. Representative polymers that form hydrogels are known to the prior art in U.S. Pat. No. 3,865,108; U.S. Pat. No. 4,002,173; U.S. Pat. No. 4,207,893; and in Handbook of Common Polymers, by Scott and Roff, published by the Chemical Rubber Co., Cleveland, Ohio. The amount of osmopolymer comprising a hydro-activated layer can be from about 5% to 100%.

The expandable layer in another manufacture can comprise an osmotically effective compound that comprises inorganic and organic compounds that exhibit an osmotic pressure gradient across a semipermeable wall against an external fluid. The osmotically effective compounds, as with the osmopolymers, imbibe fluid into the osmotic system, thereby making available fluid to push against the inner wall, i.e., in some embodiments, the barrier layer and/or the wall of the soft or hard capsule for pushing active agent from the dosage form. The osmotically effective compounds are known also as osmotically effective solutes, and also as osmagents. Osmotically effective solutes that can be used comprise magnesium sulfate, magnesium chloride, potassium sulfate, sodium sulfate, lithium sulfate, potassium acid phosphate, mannitol, urea, inositol, magnesium succinate, tartaric acid, carbohydrates such as raffinose, sucrose, glucose, lactose, sorbitol, and mixtures therefor. The amount of osmagent in can be from about 5% to 100% of the weight of the layer. The expandable layer optionally comprises an osmopolymer and an osmagent with the total amount of osmopolymer and osmagent equal to 100%. Osmotically effective solutes are known to the prior art as described in U.S. Pat. No. 4,783,337.

In certain embodiments, the dosage forms further can comprise a barrier layer. The barrier layer in certain embodiments is deformable under the pressure exerted by the expandable layer and will be impermeable (or less permeable) to fluids and materials that can be present in the expandable layer, the liquid active agent formulation and in the environment of use, during delivery of the active agent formulation. A certain degree of permeability of the barrier layer can be permitted if the delivery rate of the active agent formulation is not detrimentally effected. However, it is preferred that barrier layer not completely transport through it fluids and materials in the dosage form and the environment of use during the period of delivery of the active agent. The barrier layer can be deformable under forces applied by expandable layer so as to permit compression of capsule to force the liquid, active agent formulation from the exit orifice. In some embodiments, the barrier layer will be deformable to such an extent that it create a seal between the expandable layer and the semipermeable layer in the area where the exit orifice is formed. In that manner, the barrier layer will deform or flow to a limited extent to seal the initially, exposed areas of the expandable layer and the semipermeable layer when the exit orifice is being formed, such as by drilling or the like, or during the initial stages of operation. When sealed, the only avenue for liquid permeation into the expandable layer is through the semipermeable layer, and there is no back-flow of fluid into the expandable layer through the exit orifice.

Suitable materials for forming the barrier layer can include, for example, polyethylene, polystyrene, ethylene-vinyl acetate copolymers, polycaprolactone and Hytrel™ polyester elastomers (Du Pont), cellulose acetate, cellulose acetate pseudolatex (such as described in U.S. Pat. No. 5,024,842), cellulose acetate propionate, cellulose acetate butyrate, ethyl cellulose, ethyl cellulose pseudolatex (such as Surelease™ as supplied by 10 Colorcon, West Point, Pa. or Aquacoat™ as supplied by FMC Corporation, Philadelphia, Pa.), nitrocellulose, polylactic acid, poly-glycolic acid, polylactide glycolide copolymers, collagen, polyvinyl alcohol, polyvinyl acetate, polyethylene vinylacetate, polyethylene teraphthalate, polybutadiene styrene, polyisobutylene, polyisobutylene isoprene copolymer, polyvinyl chloride, polyvinylidene chloride-vinyl chloride copolymer, copolymers of acrylic acid and methacrylic acid esters, copolymers of methylmethacrylate and ethylacrylate, latex of acrylate esters (such as Eudragit™ supplied by RohmPharma, Darmstaat, Germany), polypropylene, copolymers of propylene oxide and ethylene oxide, propylene oxide ethylene oxide block copolymers, ethylenevinyl alcohol copolymer, polysulfone, ethylene vinylalcohol copolymer, polyxylylenes, polyalkoxysilanes, polydimethyl siloxane, polyethylene glycol-silicone elastomers, electromagnetic irradiation crosslinked acrylics, silicones, or polyesters, thermally crosslinked acrylics, silicones, or polyesters, butadiene-styrene rubber, and blends of the above.

Preferred materials can include cellulose acetate, copolymers of acrylic acid and methacrylic acid esters, copolymers of methylmethacrylate and ethylacrylate, and latex of acrylate esters. Preferred copolymers can include poly (butyl methacrylate), (2-dimethylaminoethyl)methacrylate, methyl methacrylate) 1:2:1, 150,000, sold under the trademark EUDRAGIT E; poly (ethyl acrylate, methyl methacrylate) 2:1, 800,000, sold under the trademark EUDRAGIT NE 30 D; poly (methacrylic acid, methyl methacrylate) 1:1, 135,000, sold under the trademark EUDRAGIT L; poly (methacrylic acid, ethyl acrylate) 1:1, 250,000, sold under the trademark EUDRAGIT L; poly (methacrylic acid, methyl methacrylate) 1:2, 135,000, sold under the trademark EUDRAGIT S; poly (ethyl acrylate, methyl methacrylate, trimethylammonioethyl methacrylate chloride) 1:2:0.2, 150,000, sold under the trademark EUDRAGIT RL; poly (ethyl acrylate, methyl methacrylate, trimethylammonioethyl methacrylate chloride) 1:2:0.1, 150,000, sold as EUDRAGIT RS. In each case, the ratio x:y:z indicates the molar proportions of the monomer units and the last number is the number average molecular weight of the polymer. Especially preferred are cellulose acetate containing plasticizers such as acetyl tributyl citrate and ethylacrylate methylmethylacrylate copolymers such as Eudragit NE.

The foregoing materials for use as the barrier layer can be formulated with plasticizers to make the barrier layer suitably deformable such that the force exerted by the expandable layer will collapse the compartment formed by the barrier layer to dispense the liquid, active agent formulation. Examples of typical plasticizers are as follows: polyhydric alcohols, triacetin, polyethylene glycol, glycerol, propylene glycol, acetate esters, glycerol triacetate, triethyl citrate, acetyl triethyl citrate, glycerides, acetylated monoglycerides, oils, mineral oil, castor oil and the like. The plasticizers can be blended into the material in amounts of 10-50 weight percent based on the weight of the material.

The various layers forming the barrier layer, expandable layer and semipermeable layer can be applied by conventional coating methods such as described in U.S. Pat. No. 5,324,280. While the barrier layer, expandable layer and semipermeable wall have been illustrated and described for convenience as single layers, each of those layers can be composites of several layers. For example, for particular applications it may be desirable to coat the capsule with a first layer of material that facilitates coating of a second layer having the permeability characteristics of the barrier layer. In that instance, the first and second layers comprise the barrier layer. Similar considerations would apply to the semipermeable layer and the expandable layer.

The exit orifice can be formed by mechanical drilling, laser drilling, eroding an erodible element, extracting, dissolving, bursting, or leaching a passageway former from the composite wall. The exit orifice can be a pore formed by leaching sorbitol, lactose or the like from a wall or layer as disclosed in U.S. Pat. No. 4,200,098. This patent discloses pores of controlled-size porosity formed by dissolving, extracting, or leaching a material from a wall, such as sorbitol from cellulose acetate. A preferred form of laser drilling is the use of a pulsed laser that incrementally removes material from the composite wall to the desired depth to form the exit orifice.

It will be appreciated the dosage forms described in FIGS. 1-5 are merely exemplary of a variety of dosage forms designed for and capable of achieving administration of the inventive substance(s). Those of skill in the pharmaceutical arts can identify other dosage forms that would be suitable.

### IV. METHODS OF USE

The inventive complexes, compositions, and dosage forms are useful in treating a variety of indications. In general, the number of indications treatable using the inventive complexes, compositions, and dosage forms are the same as the number of drug moieties useful in the practice of the invention. In an aspect, the invention provides a method for treating an indication, such as a disease or disorder, in a patient by administering a composition or a dosage form that comprises an inventive complex. In one embodiment, a composition comprising the complex and a pharmaceutically-acceptable vehicle is administered to the patient via oral administration. The dose administered is generally adjusted in accord with the age, weight, and condition of the patient, taking into consideration the dosage form and the desired result.

Typical doses may comprise drug moiety in an amount ranging from about 0.01 microgram of drug moiety to about 5000 mg of drug moiety, preferably ranging from about 1 microgram of drug moiety to about 2500 mg of drug moiety, more preferably ranging from about 10 micrograms of drug moiety to about 2000 mg of drug moiety, even more preferably ranging from about 100 micrograms of drug moiety to about 1500 mg of drug moiety, and still more preferably ranging from about 500 micrograms of drug moiety to about 1000 mg of drug moiety. Typical doses may comprise the inventive complex in an amount ranging from about 0.01 microgram of the inventive complex to about 5000 mg of the inventive complex, preferably ranging from about 1 microgram of the inventive complex to about 2500 mg of the inventive complex, more preferably ranging from about 10 micrograms of the inventive complex to about 2000 mg of the inventive complex, even more preferably ranging from about 100 micrograms of the inventive complex to about 1500 mg of the inventive complex, and still more preferably ranging from about 500 micrograms of the inventive complex to about 1000 mg of the inventive complex.

From the foregoing, it can be seen how various objects and features of the invention are met. A complex comprising a drug moiety ionically bound to a counter-ion may provide enhanced solubility, relative to that observed for the non-complexed drug moiety. The complex is prepared from a novel process, where the drug moiety is reacted with the counter-ion such as an alkyl sulfate salt. This reaction results in formation of a complex between the drug moiety and the counter-ion, where the two species are associated by an ionic bond.

While there has been described and pointed out features and advantages of the invention, as applied to present embodiments, those skilled in the medical art will appreciate that various modifications, changes, additions, and omissions in the method described in the specification can be made without departing from the spirit of the invention.

### V. EXAMPLES

### Example 1

### Preparation of Complex

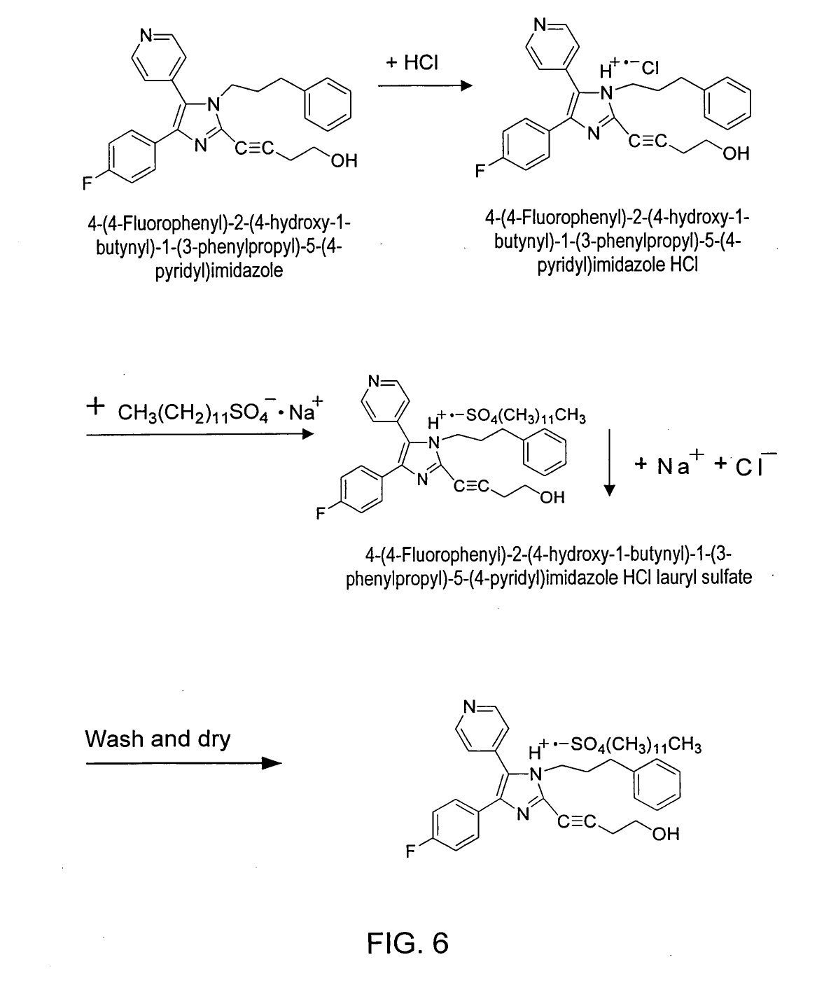

The following steps were carried out to form a lauryl sulfate complex with 4-(4-Fluorophenyl)-2-(4-hydroxy-1-butynyl)-1-(3-phenylpropyl)-5-(4-pyridyl)imidazole as shown in FIG. 6 below.

1. 2 mL 5N hydrochloric acid (10 mmol HCl) was dissolved in 100 mL deionized water at room temperature.

2. 4.255 g 4-(4-Fluorophenyl)-2-(4-hydroxy-1-butynyl)-1-(3-phenylpropyl)-5-(4-pyridyl)imidazole (10 mmol) was added to the solution in step 1. The mixture was stirred for 1 h to partially solubilize the compound at room temperature. 4-(4-Fluorophenyl)-2-(4-hydroxy-1-butynyl)-1-(3-phenylpropyl)-5-(4-pyridyl)imidazole hydrochloride was formed in solution.

3. 2.882 g sodium lauryl sulfate (10 mmol SDS) was dissolved in 40 mL deionized water at room temperature. The sodium lauryl sulfate solution was added drop by drop to the mixture in step 2. Precipitate formed immediately as sodium lauryl sulfate was added. The mixture was stirred for 24 h.

4. The mixture in step 3 was filtered with Whatman fine filter paper. The precipitate was washed three times with a total of 100 mL deionized water. The precipitate was collected, and a total 11.207 g wet product was obtained.

5. The wet product was dried at 40° C. in a vacuum oven for 22 hours. A total 6.736 g dry product was obtained. Total yield was 97.4% relative to theoretical amount calculated from the stoichiometry of 4-(4-Fluorophenyl)-2-(4-hydroxy-1-butynyl)-1-(3-phenylpropyl)-5-(4-pyridyl)imidazole and sodium lauryl sulfate.

### Example 2

### Characterization of Complex Using FTIR Spectra Shown

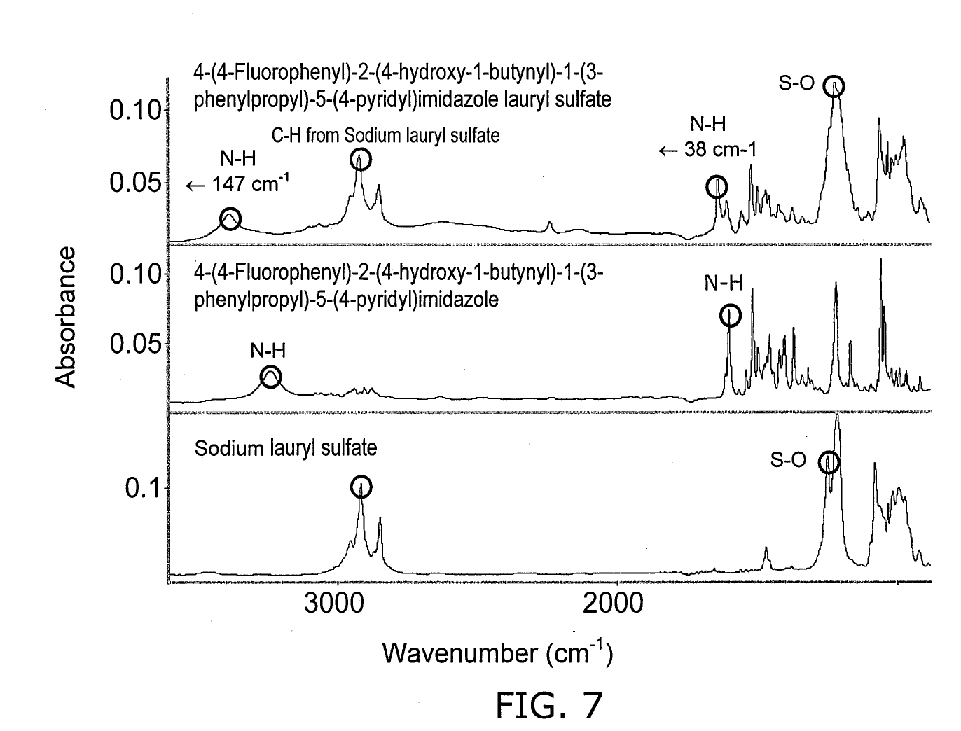

The FTIR spectra were obtained by using a Perkin-Elmer Spectrum 2000 FTIR spectrometer system that included the Attenuated Total Reflectance (ATR) accessory and liquid N2 cooled MCT (mercury cadmium telluride) detector. Spectra of complex obtained in Example 1,4-(4-Fluorophenyl)-2-(4-hydroxy-1-butynyl)-1-(3-phenylpropyl)-5-(4-pyridyl)imidazole and sodium lauryl sulfate (SDS) were obtained, and are shown in FIG. 7. Absorption at 3244 cm-1 corresponding to the N—H stretching of the drug was significantly shifted to 3391 cm-1 in the spectra of the complex. Absorption at 1600 cm-1 that is typical for the N—H deformation of the drug was significantly shifted to 1638 cm-1. The shift of bands for N—H bond indicates the protonation of the N—H groups in the resulting complex. The peak at 1250 cm-1 that is indicative of the S—O absorption in the spectra of sodium lauryl sulfate was broadened as shown in the spectra of the complex, suggesting the interaction of the drug with sulfate group of sodium lauryl sulfate. Compared with the compound, spectra of the complex shows additional absorption bands around 2925 cm-1 and 2854 cm-1 which are corresponding to the C—H stretching mode of —CH2 groups, giving further evidence for the complex formation.

### Example 3

### Characterization of Complex Using XRD

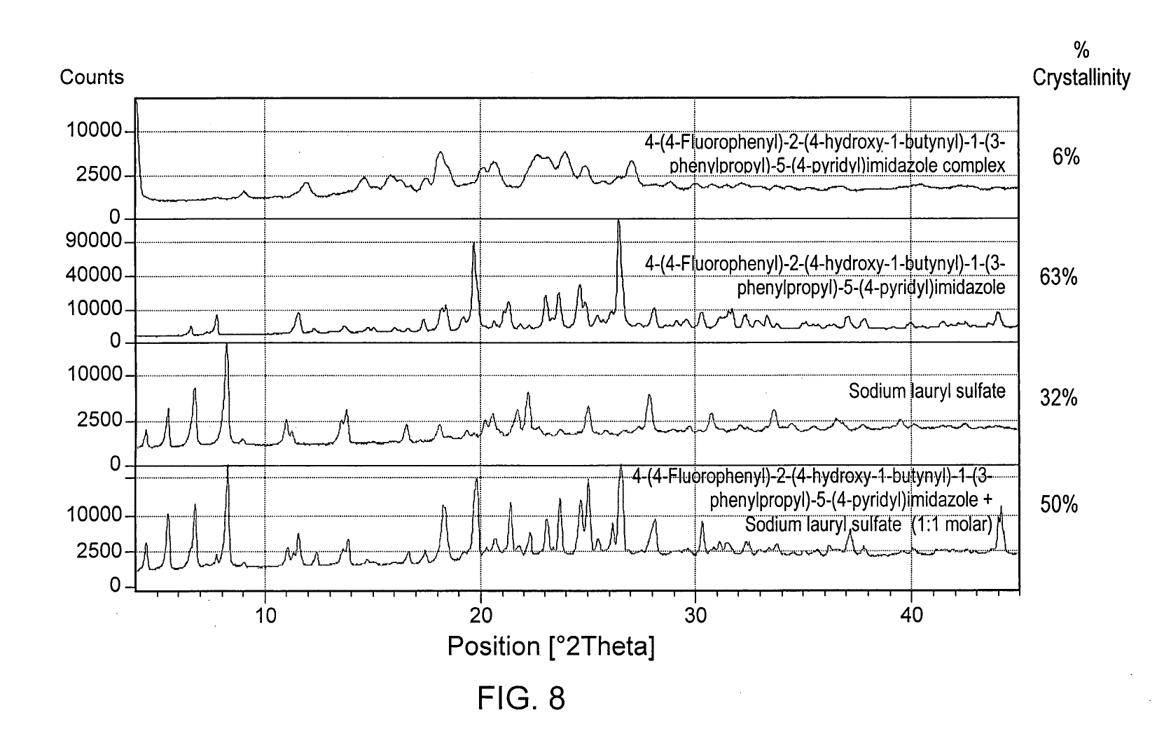

Qualitative determination of crystallinity of the complex of Example 1 was conducted with PANalytical X'Pert Pro Powder X-ray Diffraction (XRD) System. FIG. 8 shows the XRD spectra of the complex between 4-(4-Fluorophenyl)-2-(4-hydroxy-1-butynyl)-1-(3-phenylpropyl)-5-(4-pyridyl)imidazole and sodium lauryl sulfate, the drug, sodium lauryl sulfate (SDS), and physical mixture of the drug/SDS by 1:1 molar ratio. The percentage of crystallinity of the complex has been significantly reduced, with a crystallinity of 6% compared with 4-(4-Fluorophenyl)-2-(4-hydroxy-1-butynyl)-1-(3-phenylpropyl)-5-(4-pyridyl)imidazole, which possesses a crystallinity of 63%. The crystallinity of sodium lauryl sulfate was determined to be 32%, and the crystallinity of the physical mixture was determined to be 50%.

### Example 4

### Comparative Bioavailability of Complex Forms in Colonic Flushed Ligated Rat Model

An animal model commonly known as the “colonic flushed ligated” model was employed for testing formulations. Surgical preparation of a fasted anesthetized 0.3-0.5 kg Sprague-Dawley male rats proceeded as follows. A segment of proximal colon was isolated and the colon was flushed of fecal materials. The segment was ligated at both ends while a catheter was placed in the lumen and exteriorized above the skin for delivery of test formulation. The colonic contents were flushed out and the colon was returned to the abdomen of the animal.

Rats were allowed to equilibrate for approximately 1 hour after surgical preparation and prior to exposure to each test formulation. Formulations according to Table 1 were administered as a colonic bolus at dosages of 5 mg/kg of drug (see Table 1). Blood samples were obtained from the jugular catheter at 0, 15, 30, 60, 90, 120, 180 and 240 minutes after administration of the test formulation and analyzed for blood drug concentration. Formulation 1 is the dry powder of drug complex made in Example 1. The mean particle size of the power was 130 microns measured by Horiba LA 910.

Formulation 2 was prepared as follows:

1. 0.995 g Solutol HS-15 and 0.995 g drug complex according to Example 1 was added to a 20 mL vial.

2. The sample was heated in a Hotplate to raise the temperature to ˜90° C. to melt the mixture.

3. When mixture is melted it was mixed homogenously. The sample was cooled in ice water. The composition was comprised of 50% wt drug, 50% wt Solutol HS-15 The drug loading was 50%. The mean particle size of the formulation was 20 microns measured by Horiba LA 910.

4. 246 mg formulation was dispersed into 15 mL deionized water before dosing with stirring and vortexing.

Formulation 3 was prepared as follows:

1.15 g drug 4-(4-Fluorophenyl)-2-(4-hydroxy-1-butynyl)-1-(3-phenylpropyl)-5-(4-pyridyl)imidazole, 7.5 g Pluronic F108, and 7.5 g Kollidon 30 was mixed with 66 g deionized water. The mixture was milled for 3 h in a Dyno mill.

2. 130 g deinozied water was added to the milled suspension and lyophilized by using LABCONCO to remove the water. The obtained lyocake contained 50% drug, 25% Pluronic F108 and 25% Kollidon 30. The mean particle size of the formulation was 0.4 microns measured by Horiba LA 910.

3. The formulation was dosed as dry powder.

Formulation 4 was prepared as follows:

1. 50 mg 4-(4-Fluorophenyl)-2-(4-hydroxy-1-butynyl)-1-(3-phenylpropyl)-5-(4-pyridyl)imidazole and 34 mg sodium lauryl sulfate was dispersed in 10 mL deionized water as suspension. The mean particle size of the formulation is 68 microns measured by Horiba LA 910.

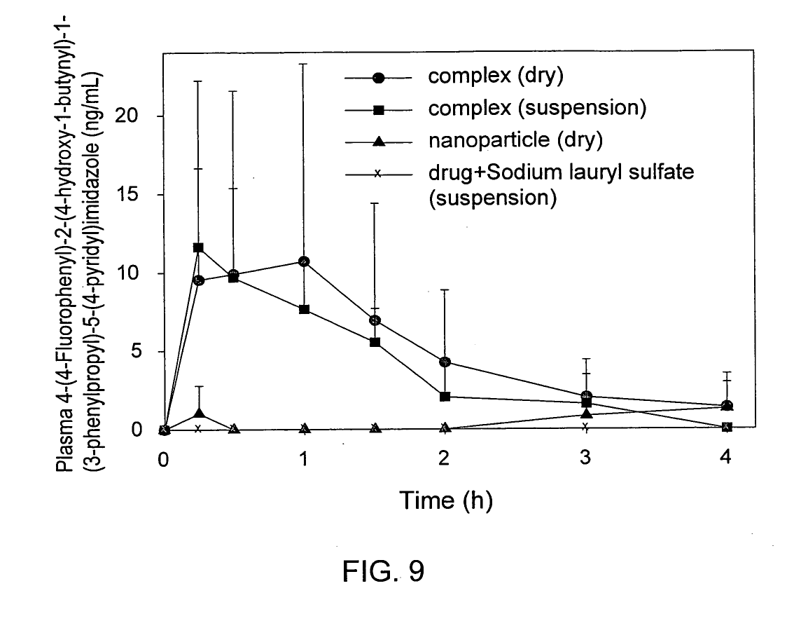

The results are shown in Table 1, and in FIG. 9

TABLE 1

| | Particle | | | | AUC | | | |
|---|---|---|---|---|---|---|---|---|
| | Size | Loading | Dose | | ng · h · kg | CV | Relative | Loading |
| Formulations | (μm) | (wt) | (mg/kg) | n | mL · mg | (%) | BA (%) | Efficiency |
| *A lauryl sulfate | 130 | 61% | 6.3 | 4 | 3.46 | 120 | 14.4 | 878 |
| complex | | | | | | | | |
| Nanoparticle: lauryl | 20 | 31% | 5 | 4 | 3.23 | 56 | 13.5 | 419 |
| sulfate/solutol HS | | | | | | | | |
| 15 = 50/50 | | | | | | | | |
| *A/F108/kollidon | 0.4 | 50% | 7.1 | 3 | 0.24 | 50 | 1 | 50 |
| 30 = 50/25/25 | | | | | | | | |
| *A+SDS (1:1 molar) | 68 | 60% | 4.9 | 6 | 0 | 0 | 0 | 0 |

Loading Efficiency = (loading %) × (rBA %)
*A = 4-(4-Fluorophenyl)-2-(4-hydroxy-1-butynyl)-1-(3-phenylpropyl)-5-(4-pyridyl)imidazole

### Example 5

### Dissolution Testing of the Complex

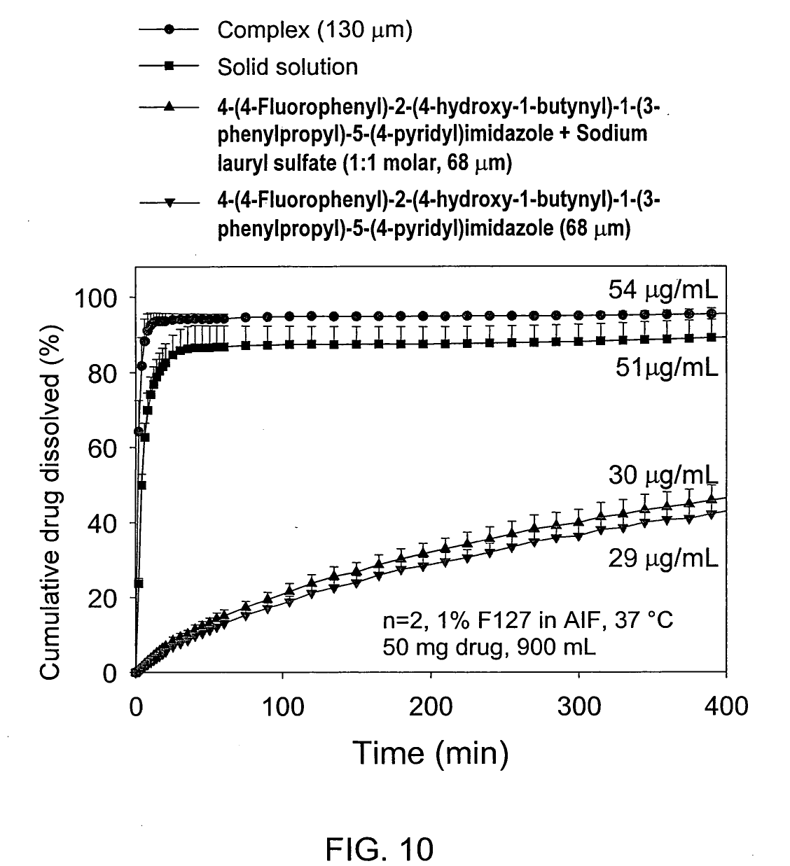

Dissolution studies were performed on the Distek 5100 (USP apparatus 2 paddle tester) in 900 mL artificial intestinal fluid (AIF, pH=6.8) with 1% (by weight) of Pluronic F127. The temperature of the dissolution medium was maintained at 37° C. and the paddle speed was 100 rpm. The concentration of the compound was measured with online UV spectroscopy at 270 nm. FIG. 10 shows the dissolution profiles of the following formulations:

1. Complex according to Example 1, mean particle size of 130 mm.

2. Solid solution of 4-(4-Fluorophenyl)-2-(4-hydroxy-1-butynyl)-1-(3-phenylpropyl)-5-(4-pyridyl)imidazole in Kollidon VA64=25/75 (by weight), granule size of <425 mm.

3. Physical mixture of 4-(4-Fluorophenyl)-2-(4-hydroxy-1-butynyl)-1-(3-phenylpropyl)-5-(4-pyridyl)imidazole and sodium lauryl sulfate (1:1 molar ratio), mean particle size of the compound of 68 mm.

4. 4-(4-Fluorophenyl)-2-(4-hydroxy-1-butynyl)-1-(3-phenylpropyl)-5-(4-pyridyl)imidazole, mean particle size of 68 mm.

As shown in FIG. 10, both complex and solid solution showed faster dissolution rate and higher solubility compared with raw drug and physical mixture of drug and sodium lauryl sulfate.

### Example 6

### Biovailability of Complex

Using the procedures outlined in Example 4, the bioavailability of the formulations listed below in Table 2 was determined in the Rat Flushed Ligated Colon model.

Formulation 1 is a solid solution prepared as:

1. A mixture of 25 wt % 4-(4-Fluorophenyl)-2-(4-hydroxy-1-butynyl)-1-(3-phenylpropyl)-5-(4-pyridyl)imidazole and 75 wt % Kollidon VA64 was added to a 10 mL polymer mixer. Raise the temperature to 160° C. to melt the mixture and cool down to the room temperature.

2. The above material was passed through a 40 mesh screen.

3. The formulation was dosed to the rats as a powder.

Formulation 2 was prepared as:

1. 4 g Solutol HS-15 and 1.5 g 4-(4-Fluorophenyl)-2-(4-hydroxy-1-butynyl)-1-(3-phenylpropyl)-5-(4-pyridyl)imidazole was added to a 20 mL vial.

2. The sample was heated in a Hotplate to raise the temperature to −90° C. to solubilize the drug. Then 4.5 g Kollidon VA 64 was added to the vial. The temperature was raised to 160° C.

3. The mixture of step 3 was melted and mixed homogenously. The sample was cooled in ice water.

4. The composition was comprised of 15% wt 4-(4-Fluorophenyl)-2-(4-hydroxy-1-butynyl)-1-(3-phenylpropyl)-5-(4-pyridyl)imidazole, 40% wt Solutol HS-15 and 45% wt Kollidon VA 64.

5.500 mg of the above formulation was dispersed into 15 mL deionized water before dosing with stirring and vortexing.

Formulation 3 is the same as the formulation 3 in example 4.

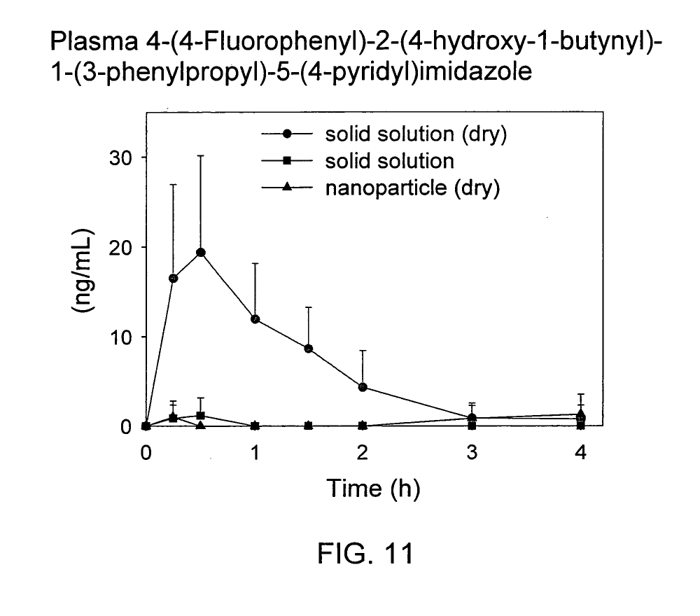

The results are shown in Table 2 and FIG. 11.

| | Particle | | | | AUC | | |
|---|---|---|---|---|---|---|---|
| | Size | Loading | Dose | | ng · h · kg | CV | Relative |
| Formulations | (μm) | (wt) | (mg/kg) | n | mL · mg | (%) | BA (%) |
| *A/kollidon VA64 = 25/75 | <425 | 25% | 6.3 | 4 | 4.05 | 62 | 16.9 |
| *A/kollidon VA64/solutol HS | 4 | 15% | 5 | 4 | 0.42 | 145 | 1.75 |
| 15 = 15/45/40 | | | | | | | |
| Naoparticle: *A/F108/kollidon | 0.4 | 50% | 7.1 | 3 | 0.24 | 50 | 1 |
| 30 = 50/25/25 | | | | | | | |

*A = 4-(4-Fluorophenyl)-2-(4-hydroxy-1-butynyl)-1-(3-phenylpropyl)-5-(4-pyridyl)imidazole

### Example 7

### Oral Dosage Form Comprising Complex (Prophetic)

The drug lauryl sulfate complex layer in the dosage form is prepared as follows: First, 8.24 g of drug lauryl sulfate complex prepared according to Example 1, 0.26 g polyethylene oxide N-80, 0.8 g poloxamer 188 NF, 0.3 g croscarmellose sodium, and 0.3 g povidone K29-32 are dry blended in a conventional blender for 20 minutes to yield a homogenous blend. Next, anhydrous ethanol is added slowly to the blend with continuous mixing for 5 minutes. The blended wet composition is passed through a 16 mesh screen and dried overnight at room temperature. Then, the dry granules are passed through a 16 mesh screen and 0.1 g magnesium stearate is added and all the dry ingredients are dry blended. The composition is comprised of 82.4 wt % drug complex, 2.6% wt polyethylene oxide N-80, 8% wt poloxamer 188 NF, 3% wt croscarmellose sodium, and 3% wt povidone K29-32 and 1 wt % magnesium stearate.

A push layer comprised of an osmopolymer hydrogel composition is prepared as follows. First, 58.67 g of pharmaceutically acceptable polyethylene oxide comprising a 7,000,000 molecular weight, 5 g Carbopol 974P, 30 g sodium chloride, 1 g ferric oxide, and 5 g of hydroxypropylmethylcellulose of 9,200 molecular weight are separately screened through a 40 mesh screen. The screened ingredients are mixed for 20 minutes to produce a homogenous blend. Next, 25 mL of denatured anhydrous alcohol is added slowly to the blend with continuous mixing. Then, 0.08 g of butylated hydroxytoluene is added followed by more blending. The freshly prepared granulation is passed through a 16 mesh screen and allowed to dry for 20 hours at room temperature (ambient). The dried ingredients are passed through a 20 mesh screen and 0.25 g of magnesium stearate is added and all the ingredients are blended for 5 minutes. The final composition is comprised of 58.67 wt % of polyethylene oxide, 5 wt % Carbopol 974P, 30.00 wt % sodium chloride, 1.00 wt % ferric oxide, 5.00 wt % hydroxypropylmethylcellulose, 0.08 wt % butylated hydroxytoluene and 0.25 wt % magnesium stearate.

The bi-layer dosage form is prepared as follows. First, 198 mg of the drug layer granules is added to a punch and die set and tamped. Then 99 mg of the push layer granules is added and the two layers compressed under a compression force of 1.0 ton (1000 kg) into a 9/32 inch (0.714 cm) diameter punch die set, forming an intimate bi-layered core (tablet).

A semipermeable wall-forming composition is prepared comprising 80.0 wt % cellulose acetate having a 39.8% acetyl content and 20.0% polyoxyethylene-polyoxypropylene copolymer having a molecular weight of 7680-9510 by dissolving the ingredients in acetone in a 80:20 wt/wt composition to make a 5.0% solids solution. Placing the solution container in a warm water bath during this step accelerates the dissolution of the components. The wall-forming composition is sprayed onto and around the bi-layered core to provide a 60 to 80 mg thickness semi-permeable wall.

Next, a 40 mil (1.02 mm) exit orifice is laser drilled in the semipermeable walled bi-layered tablet to provide contact of the drug lauryl sulfate complex layer with the exterior of the delivery device. The dosage form is dried to remove any residual solvent and water.

### Example 8

### Preparation of Phenazopyridine Lauryl Sulfate Complex (Prophetic)

The following steps are carried out to form a lauryl sulfate complex of Phenazopyridine

1. 2 mL 5N hydrochloric acid (10 mmol HCl) is dissolved in 100 mL deionized water at room temperature.

2. 2.132 g Phenazopyridine (10 mmol) is added to the solution in step 1. The mixture is stirred to partially solubilize the compound at room temperature. Phenazopyridine hydrochloride is formed in solution.

3. 2.882 g sodium lauryl sulfate (10 mmol SDS) is dissolved in 40 mL deionized water at room temperature. The sodium lauryl sulfate solution is added drop by drop to the mixture in step 2. Precipitate is formed as sodium lauryl sulfate is added. The mixture is stirred for 3 h.

4. The mixture in step 3 is filtered with Whatman fine filter paper. The precipitate is washed three times with a total of 100 mL deionized water. The precipitate is collected.

5. The wet product is dried at 40° C. in a vacuum oven for 22 hours. A total maximum 4.8 g dry product can be obtained based on the theoretical amount calculated from the stoichiometry of Phenazopyridine and sodium lauryl sulfate.

### Example 9

### Matrix Dosage Form Comprising Inventive Complex (Prophetic)

A matrix dosage form according to the present invention is prepared as follows. 200 grams of the complex prepared as in Example 1, 25 grams of hydroxypropyl methylcellulose having a number average molecular weight of 9,200 grams per mole, and 15 grams of hydroxypropyl methylcellulose having a molecular weight of 242,000 grams per mole, are passed through a screen having a mesh size of 40 wires per inch. The celluloses each have an average hydroxyl content of 8 weight percent and an average methoxyl content of 22 weight percent. The resulting sized powders are tumble mixed. Anhydrous ethyl alcohol is added slowly to the mixed powders with stirring until a dough consistency is produced. The damp mass is then extruded through a 20 mesh screen and air dried overnight. The resulting dried material is re-screened through a 20 mesh screen to form the final granules. 2 grams of the tabletting lubricant, magnesium stearate, which are sized through an 80 mesh screen, are then tumbled into the granules.

393 mg of the resulting granulation is placed in a die cavity having an inside diameter of 9/32 inch and compressed with deep concave punch tooling using a pressure head of 2 tons. This forms a longitudinal capsule core having an overall length, including the rounded ends, of 0.691 inch. The cylindrical body of the capsule, from tablet land to tablet land, span a distance of 12 mm. Each core contains a unit dose of the complex according to Example 1 of 325 mg, (200 mg equivalent of the compound).

### Example 10

### Modified Matrix Dosage Form (Prophetic)

A modified matrix dosage form according to the present invention is prepared as follows. A matrix delivery system according to Example 10 above is prepared to form a core. The core contains a unit dose of the complex according to Example 1 of 325 mg. Rings of polyethylene having an inside diameter of 9/32 inch, a wall thickness of 0.013 inch, and a width of 2 mm are then fabricated. These rings, or bands, are press fitted onto the core to complete the dosage form.

What is claimed is:

1. A substance comprising:
a complex comprising a drug moiety ionically bound to a counter-ion;
wherein a solubility of the complex is greater than a solubility of the drug moiety.

2. The substance of claim 1, wherein the drug moiety comprises at least one of a(n) acidic, basic, or zwitterionic structural element, or a(n) acidic, basic, or zwitterionic residual structural element.

3. The substance of claim 2, wherein a pKa of an acidic structural element or acidic residual structural element, is less than about 7.0.

4. The substance of claim 3, wherein a pKa of an acidic structural element or acidic residual structural element, is less than about 6.0.

5. The substance of claim 2, wherein a zwitterionic structural element or a zwitterionic residual structural element comprises the acidic structural element or acidic residual structural element.

6. The substance of claim 2, wherein a pKa of a basic structural element or basic residual structural element is greater than about 7.0.

7. The substance of claim 6, wherein a pKa of an basic structural element or acidic residual structural element, is greater than about 8.0.

8. The substance of claim 2, wherein a zwitterionic structural element or a zwitterionic residual structural element comprises the basic structural element or basic residual structural element.

9. The substance of claim 1, wherein the counter-ion comprises a saturated or unsaturated alkyl sulfate or a salt thereof

10. The substance of claim 9, wherein the counter-ion comprises sodium octyl sulfate, sodium decyl sulfate, sodium lauryl sulfate, or sodium tetradecyl sulfate.

11. A composition comprising the substance of claim 1 and a pharmaceutically acceptable carrier.

12. A dosage form comprising the composition of claim 11.

13. A method of preparing a substance comprising:
providing a drug moiety;
mixing the drug moiety into a solvent that has been titrated to a pH as follows:
(a) for acidic structural elements or acidic residual structural elements, the the environmental pH is less than or equal to about the pKa of the drug moiety minus two pH units,
(b) for basic structural elements or basic residual structural elements, the environmental pH is greater than or equal to about the pKa of the drug plus two pH units;
adding a counter-ion to the mixture of the drug moiety and the solvent;
forming a complex that comprises the drug moiety and the counter-ion; and
recovering the complex.

14. The method of claim 13, wherein the drug moiety comprises at least one of a(n) acidic, basic, or zwitterionic structural element, or a(n) acidic, basic, or zwitterionic residual structural element.

15. The method of claim 14, wherein a pKa of an acidic structural element or acidic residual structural element, is less than about 7.0.

16. The method of claim 15, wherein a pKa of an acidic structural element or acidic residual structural element, is less than about 6.0.

17. The method of claim 14, wherein a zwitterionic structural element or a zwitterionic residual structural element comprises the acidic structural element or acidic residual structural element.

18. The method of claim 14, wherein a pKa of a basic structural element or basic residual structural element is greater than about 7.0.

19. The method of claim 18, wherein a pKa of an basic structural element or acidic residual structural element, is greater than about 8.0.

20. The method of claim 14, wherein a zwitterionic structural element or a zwitterionic residual structural element comprises the basic structural element or basic residual structural element.

21. The method of claim 13, wherein the counter-ion comprises a saturated or unsaturated alkyl sulfate or a salt thereof

22. The method of claim 21, wherein the counter-ion comprises sodium octyl sulfate, sodium decyl sulfate, sodium lauryl sulfate, or sodium tetradecyl sulfate.

23. A composition that comprises a substance made according to the method of claim 13, and a pharmaceutically acceptable carrier.

24. A dosage form comprising the composition of claim 23.

25. A method comprising:
providing a complex comprising a drug moiety ionically bound to a counter-ion; wherein a solubility of the complex is greater than a solubility of the drug moiety; and
administering the complex to a patient.

26. The method of claim 25, wherein the complex is orally administered to a patient.

27. The method of claim 25, wherein the drug moiety comprises at least one of a(n) acidic, basic, or zwitterionic structural element, or a(n) acidic, basic, or zwitterionic residual structural element.

28. The method of claim 27, wherein a pKa of an acidic structural element or acidic residual structural element, is less than about 7.0.

29. The method of claim 28, wherein a pKa of an acidic structural element or acidic residual structural element, is less than about 6.0.

30. The method of claim 27, wherein a zwitterionic structural element or a zwitterionic residual structural element comprises the acidic structural element or acidic residual structural element.

31. The method of claim 27, wherein a pKa of a basic structural element or basic residual structural element is greater than about 7.0.

32. The method of claim 31, wherein a pKa of an basic structural element or acidic residual structural element, is greater than about 8.0.

33. The method of claim 27, wherein a zwitterionic structural element or a zwitterionic residual structural element comprises the basic structural element or basic residual structural element.

34. The method of claim 25, wherein the counter-ion comprises a saturated or unsaturated alkyl sulfate or a salt thereof

35. The method of claim 34, wherein the counter-ion comprises sodium octyl sulfate, sodium decyl sulfate, sodium lauryl sulfate, or sodium tetradecyl sulfate.
